(19) United States
(12) Patent Application Publication
(10) Pub. No.: US 2026/0345766 A1
(43) Pub. Date: Nov. 5, 2026

(54) RECOMBINANT ROTAVIRUSES AND METHODS AND SYSTEMS FOR PRODUCING THE SAME

(71) Applicant: THE TRUSTEES OF INDIANA UNIVERSITY, Bloomington, IN (US)

(72) Inventors: John T. PATTON, Bloomington, IN (US); Asha A. PHILIP, Bloomington, IN (US); Chantal A. AGBEMABIESE, Bloomington, IN (US)

(21) Appl. No.: 19/116,157

(22) PCT Filed: Oct. 9, 2023
(86) PCT No.: PCT/US2023/076397

§ 371 Date: Mar. 27, 2025

Related U.S. Application Data
(60) Provisional application No. 63/414,283, filed on Oct. 7, 2022.
(60) Provisional application No. 63/510,958, filed on Jun. 29, 2023.

(51) Int. Cl. A61K 39/15; A61K 39/00; A61K 39/295; A61P 31/14; C12N 5/071; C12N 7/02; C12N 15/11

(52) U.S. Cl. CPC A61K 39/15; A61K 39/295; A61P 31/14; C12N 5/0686; C12N 7/025; C12N 15/11; A61K 2039/53; A61K 2039/70; C12N 2510/04; C12N 2720/12322; C12N 2720/12334; C12N 2720/12352; C12N 2830/34

(57) ABSTRACT

Disclosed are novel recombinant rotaviruses and methods of making the same, systems for generating the same, as well as methods of using the same to elicit an immune response in a subject or to vaccinate a subject against rotavirus infection and infection.

### CROSS-REFERENCE TO RELATED APPLICATIONS

This application claims priority to U.S. Provisional Patent Application Nos. 63/414,283, filed Oct. 7, 2022, and 63/510,958, filed Jun. 29, 2023, the entire content of each of which is incorporated by reference.

### STATEMENT REGARDING FEDERALLY SPONSORED RESEARCH OR DEVELOPMENT

This invention was made with government support under AI144881 awarded by the National Institutes of Health. The Government has certain rights in the invention.

### SEQUENCE LISTING

A Sequence Listing accompanies this application and is submitted as an xml file of the sequence listing named “144578_00383.xml” which is 55,776 bytes in size and was created on Oct. 9, 2023. The sequence listing is electronically submitted via Patent Center and is incorporated by reference in its entirety.

### BACKGROUND

Reverse genetics systems have been developed for several rotavirus strains, including simian strain SA11, rhesus strain RRV, murine-like strain rD6/2-2g, bovine RF, avian strain PO-13, human G1P[8] strain KU, human G1P[8] strain CDC-9, and human G4P[8] strain Odelia. The most widely used rotavirus vaccine (Rotarix) is made by GSK and is formulated from the human G1P[8] strain RIX4414. In fact, ~75% of children that receive rotavirus vaccinations, receive Rotarix. The demand for rotavirus vaccine doses is expected to reach nearly 300 million by 2030. Therefore, there exists a need in the art for novel recombinant rotaviruses.

### SUMMARY

In one aspect of the current disclosure, compositions are provided. In some embodiments, the compositions comprise a polynucleotide comprising a sequence encoding a recombinant rotavirus protein, wherein the polynucleotide encodes a positive sense viral transcript, wherein the recombinant rotavirus protein is an RIX4414 strain rotavirus protein.

In an aspect of the current disclosure, collections of polynucleotides are provided. In some embodiments, each of the polynucleotides in the collection comprises a sequence encoding at least one RIX4414 strain rotavirus protein selected from VP1, VP2, VP3, VP4, VP6, VP7, NSP1, NSP2, NSP3, NSP4, and NSP5, wherein the polynucleotides of the collection encode each of the VP1, VP2, VP3, VP4, VP6, VP7, NSP1, NSP2, NSP3, NSP4, and NSP5 proteins, wherein each of the sequences encoding one rotavirus protein are operably linked to a promoter.

In aspect of the current disclosure, infectious particles are provided. In some embodiments, the infectious particles comprise a sequence encoding a recombinant rotavirus protein, wherein the polynucleotide encodes a positive sense viral transcript, wherein the recombinant rotavirus protein is an RIX4414 strain rotavirus protein.

In some embodiments the infectious particles are made by transfecting a cell with a collection of polynucleotides, each of the polynucleotides in the collection comprising a sequence encoding at least one RIX4414 strain rotavirus protein selected from VP1, VP2, VP3, VP4, VP6, VP7, NSP1, NSP2, NSP3, NSP4, and NSP5, wherein the polynucleotides of the collection encode each of the VP1, VP2, VP3, VP4, VP6, VP7, NSP1, NSP2, NSP3, NSP4, and NSP5 proteins, wherein each of the sequences encoding one rotavirus protein are operably linked to a promoter.

In an aspect of the current disclosure, pharmaceutical compositions are provided. In some embodiments, the pharmaceutical compositions comprise an infectious particle comprising a sequence encoding a recombinant rotavirus protein, wherein the polynucleotide encodes a positive sense viral transcript, wherein the recombinant rotavirus protein is an RIX4414 strain rotavirus protein.

In an aspect of the current disclosure, methods are provided. In some embodiments, the methods comprise: administering a pharmaceutical composition comprising an infectious particle comprising a sequence encoding a recombinant rotavirus protein, wherein the polynucleotide encodes a positive sense viral transcript, wherein the recombinant rotavirus protein is an RIX4414 strain rotavirus protein to a subject.

In an aspect of the current disclosure, methods of eliciting an immune response to one or more microorganism in a subject are provided. In some embodiments, the methods comprise: administering a pharmaceutical composition comprising an infectious particle comprising a sequence encoding a recombinant rotavirus protein, wherein the polynucleotide encodes a positive sense viral transcript, wherein the recombinant rotavirus protein is an RIX4414 strain rotavirus protein to a subject to elicit an immune response to the one or more microorganism.

In some embodiments, the methods comprise administering a pharmaceutical composition comprising an infectious particle comprising a sequence encoding a recombinant rotavirus protein, wherein the polynucleotide encodes a positive sense viral transcript, wherein the recombinant rotavirus protein is an RIX4414 strain rotavirus protein to a subject to elicit an immune response in the subject to a pathogen or vaccinate the subject against one or more pathogens.

In an aspect of the current disclosure, methods of vaccinating a subject against one or more pathogens are provided. In some embodiments, the methods comprise: administering a pharmaceutical composition comprising an infectious particle comprising a sequence encoding a recombinant rotavirus protein, wherein the polynucleotide encodes a positive sense viral transcript, wherein the recombinant rotavirus protein is an RIX4414 strain rotavirus protein to a subject to vaccinate a subject against the one or more pathogens.

In an aspect of the current disclosure, cells are provided. In some embodiments, the cells cell comprising a composition comprising a sequence encoding at least one RIX4414 strain rotavirus protein selected from VP1, VP2, VP3, VP4, VP6, VP7, NSP1, NSP2, NSP3, NSP4, and NSP5, wherein the polynucleotides of the collection encode each of the VP1, VP2, VP3, VP4, VP6, VP7, NSP1, NSP2, NSP3, NSP4, and NSP5 proteins, wherein each of the sequences encoding one rotavirus protein are operably linked to a promoter or an infectious particle comprising a sequence encoding a recombinant rotavirus protein, wherein the polynucleotide encodes a positive sense viral transcript, wherein the recombinant rotavirus protein is an RIX4414 strain rotavirus protein.

In an aspect of the current disclosure, methods of generating a rotavirus in vitro are provided. In some embodiments, the methods comprise: introducing a composition comprising a sequence encoding a recombinant rotavirus protein, wherein the polynucleotide encodes a positive sense viral transcript, wherein the recombinant rotavirus protein is an RIX4414 strain rotavirus protein; allowing the cell to express one or more rotavirus proteins selected from VP1, VP2, VP3, VP4, VP6, VP7, NSP1, NSP2, NSP3, NSP4, and NSP5; incubating the cells for a sufficient time to produce rotavirus; and harvesting virus produced by the cells to generate the rotavirus in vitro.

In some embodiments, the methods of generating a rotavirus in vitro comprise: introducing a collection of polynucleotides, each of the polynucleotides in the collection comprising a sequence encoding at least one RIX4414 strain rotavirus protein selected from VP1, VP2, VP3, VP4, VP6, VP7, NSP1, NSP2, NSP3, NSP4, and NSP5, wherein the polynucleotides of the collection encode each of the VP1, VP2, VP3, VP4, VP6, VP7, NSP1, NSP2, NSP3, NSP4, and NSP5 proteins, wherein each of the sequences encoding one rotavirus protein are operably linked to a promoter into a cell; incubating the cells for a sufficient time to produce rotavirus; and harvesting virus produced by the cells to generate the rotavirus in vitro.

In an aspect of the current disclosure, systems for generating recombinant rotavirus are provided. In some embodiments, the systems comprise: (a) a composition comprising a sequence encoding a recombinant rotavirus protein, wherein the polynucleotide encodes a positive sense viral transcript, wherein the recombinant rotavirus protein is an RIX4414 strain rotavirus protein; and (b) cells capable of expressing the compositions of (a).

In some embodiments, the systems for generating recombinant rotavirus comprise: (a) a comprising a sequence encoding a recombinant rotavirus protein, wherein the polynucleotide encodes a positive sense viral transcript, wherein the recombinant rotavirus protein is an RIX4414 strain rotavirus protein; and (b) cells capable of expressing the collection of (a).

### BRIEF DESCRIPTION OF THE FIGURES

FIG. 1 shows a polyacrylamide gel demonstrating the size of genome segments of rotavirus isolates. Lane 1: recombinant RIX4414 (human G1P[8]). Lane 2: recombinant reassortant of RIX4414 & Odelia. Lane 3: recombinant reassortant of RIX4414 & Odelia. Lane 4: recombinant Odelia (human G4P[8]).

FIGS. 2A and 2B show recovery of recombinant RIX4414-like virus by reverse genetics. Genome segments purified by Trizol extraction from (A) recombinant SA11 virus (rSA11wt), RIX4414-like virus (rRIX4414L), a reassortant strain of RIX4414-like virus and Odelia (rOdelia), and rOdelia, and (B) rRIX4414L and RIX4414 derived from RV1 vaccine (vRIX4414), were resolved by electrophoresis on 10% polyacrylamide gels and stained with ethidium bromide. Positions of genome segments 1-11 of (A) rSA11wt and (B) rRIX4414L are indicated. The recombinant viruses rSA11wt and rOdelia were generated as described previously (8, 10). For the reassortant strain rRIX4414L/rOdelia, segments 1, 2, 3, 5 and 6 derive from Odelia, the remainder from RIX4414 (8).

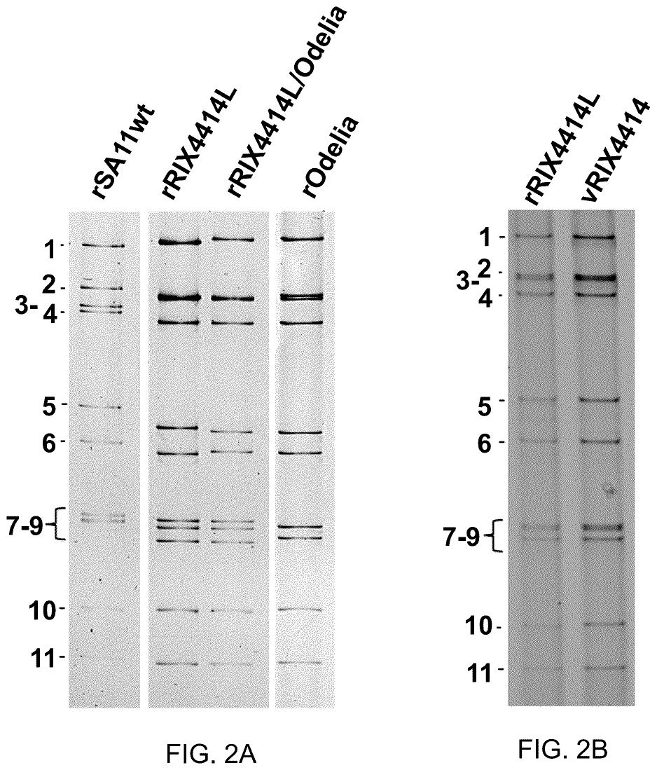

### DETAILED DESCRIPTION

The introduction of rotavirus (RV) vaccines has reduced the incidence of severe gastroenteritis in young children in many countries. As an approach for generating rotavirus-based vaccines that provide protection against multiple pathogens, the inventors have examined the possibility of using RV as an expression vector of foreign proteins. The approach incorporates a 2 A stop-restart translation element and a foreign ORF in the RV segment 7 (NSP3) RNA such that segment gains the capacity to encode two separate proteins: NSP3 and a heterologous protein. To apply this technology, the inventors developed a reverse genetics system for the human G1P[8] rotavirus strain (RIX4414) (formulated into the widely used ROTARIX vaccine). Through study of RIX4414 and RIX4414/SA11 reassortant viruses, the inventors determined that the RIX4414 segment 7 RNA can be re-engineered to express NSP3 and capsid proteins of other RNA viruses, including norovirus (NoV) and SARS-CoV-2. For example, insertion of 0.9 and 1.6 kb NoV sequences into RIX4414 segment 7 allows the expression of NoV P and VP1 capsid proteins, respectively. Moreover, insertion of 0.8 and 2.1 kb SARS-CoV-2 sequences into RIX4414 segment 7 allows the expression of the RBD and S1 domains of the SARS-CoV-2 spike protein, respectively. Further analysis of these and other recombinant RVs indicates that segment 7 sequence insertions up to 1.1 kb are genetically stable. These results indicate that RIX4414 may be used as a potential childhood vaccine vector, allowing the development of combination vaccines against NoV and other pathogens.

The inventors generated a novel reverse genetics system for producing recombinant attenuated rotavirus, e.g., the rotavirus strain RIX4414 (more commonly known as the strain found in ROTARIX live-attenuated rotavirus vaccine). In addition, the inventors disclose herein that the novel reverse genetics system can be used to generate recombinant rotavirus comprising heterologous polynucleotide sequences, e.g., polynucleotide sequences encoding additional antigens, e.g., norovirus antigens. The inventors believe that the disclosed compositions, pharmaceutical compositions, infectious particles, methods, and systems may allow the combined vaccination against the two most common causes of severe gastroenteritis in children.

### DEFINITIONS

The disclosed subject matter may be further described using definitions and terminology as follows. The definitions and terminology used herein are for the purpose of describing particular embodiments only and are not intended to be limiting.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

A “subject in need thereof” as utilized herein may refer to a subject at risk for rotavirus infection. In some embodiments, the disclosed compositions, methods, and infectious particles comprise heterologous polynucleotides that encode for additional, non-rotaviral, proteins or peptides. Therefore, in some embodiments, a subject in need thereof may refer to a subject at risk of rotaviral infection and/or infection by another pathogen, wherein the heterologous polynucleotide encodes an antigen, e.g., protein or peptide, from the pathogen which is not a rotavirus.

The term “subject” may be used interchangeably with the terms “individual” and “patient” and includes human and non-human mammalian subjects.

The phrases “% sequence identity,” “percent identity,” or “identity” refer to the percentage of amino acid residue matches between at least two amino acid sequences aligned using a standardized algorithm. Methods of amino acid sequence alignment are well-known. Some alignment methods take into account conservative amino acid substitutions. Such conservative substitutions, explained in more detail below, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastp,” that is used to align a known amino acid sequence with other amino acids sequences from a variety of databases.

Nucleic acids, proteins, and/or other compositions described herein may be purified. As used herein, “purified” means separate from the majority of other compounds or entities, and encompasses partially purified or substantially purified. Purity may be denoted by a weight by weight measure and may be determined using a variety of analytical techniques such as but not limited to mass spectrometry, HPLC, etc.

Polypeptide sequence identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

As used herein, the term “polypeptide,” protein” and “peptide” are used interchangeably and refer to a polymer of 3 or more amino acids. Thus, for example, a protein may include two proteins that are joined (fused) together. Moreover, a protein may refer to a portion or fragment of a protein, e.g., SARS-CoV-2 S1 protein, which is a fragment of the SARS-CoV-2 surface glycoprotein or “S” protein.

The terms “nucleic acid” and “nucleic acid molecule,” as used herein, refer to a compound comprising a nucleobase and an acidic moiety, e.g., a nucleoside, a nucleotide, or a polymer of nucleotides. Nucleic acids generally refer to polymers comprising nucleotides or nucleotide analogs joined together through backbone linkages such as but not limited to phosphodiester bonds. Nucleic acids include deoxyribonucleic acids (DNA) and ribonucleic acids (RNA) such as messenger RNA (mRNA), transfer RNA (tRNA), etc. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or include non-naturally occurring nucleotides or nucleosides. Furthermore, the terms “nucleic acid,” “DNA,” “RNA,” and/or similar terms include nucleic acid analogs, i.e. analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides, (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadeno sine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The term “hybridization”, as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions”. Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning-A Laboratory Manual, Cold Spring Harbor Laboratory, Cold Spring Harbor, New York; Wetmur, 1991, Critical Review in Biochem. and Mol. Biol. 26(3/4):227-259; and Owczarzy et al., 2008, Biochemistry, 47: 5336-5353, which are incorporated herein by reference).

### Recombinant Rotavirus Compositions

Accordingly, in a first aspect, compositions comprising a polynucleotide comprising a sequence encoding a recombinant rotavirus protein, wherein the polynucleotide encodes a positive sense viral transcript are disclosed herein.

As used herein, “RIX4414” refers to a strain of live-attenuated rotavirus. In contrast, the instant disclosure provides “recombinant RIX4414” rotaviruses produced by the disclosed reverse genetics systems, which are distinguished from the RIX4414 strain.

As used herein, an “RIX4414 strain rotavirus protein” refers to a protein derived from the RIX4414 strain of rotavirus.

In some embodiments, the polynucleotides are operably linked to a promoter to allow the expression of said polynucleotides in a cell, e.g., a mammalian cell.

As used herein, “operably linked” refers to a functional relationship between two or more nucleic acid (e.g., DNA) segments. Typically, it refers to the functional relationship of transcriptional regulatory element (promoter) to a transcribed sequence. For example, a promoter is operably linked to a coding sequence if it stimulates or modulates the transcription of the coding sequence in an appropriate cell. Generally, promoter transcriptional regulatory elements that are operably linked to a sequence are physically contiguous to the transcribed sequence, i.e., they are cis acting. However, some transcriptional regulatory elements, such as enhancers, need not be physically contiguous or located in close proximity to the coding sequences whose transcription they enhance. Exemplary promoters include a T7 bacteriophage promoter (SEQ ID NO: 14) and a T3 bacteriophage promoter (SEQ ID NO: 15). A suitable promoter may be chosen by from promoters known in the art. In some embodiments, the cells are mammalian cells and are selected from MA-104 cells, Vero cells and BHK-1 cells.

In some embodiments, the polynucleotides comprise a sequence encoding a rotaviral protein, i.e., are selected from a sequence encoding rotavirus VP1, VP2, VP3, VP4, VP6, VP7, NSP1, NSP2, NSP3, NSP4, and NSP5. The sequences encoding the RIX4414 strain VP1, VP2, VP3, VP4, VP6, VP7, NSP1, NSP2, NSP3, NSP4, and NSP5 proteins are provided herein as SEQ ID NOs: 1-11, respectively. Thus, the inventors contemplate that the disclosed compositions may comprise a polynucleotide comprising any one of SEQ ID NOs: 1-11, or functional variants thereof (e.g., nucleic acid sequence variants that encode the same amino acid due to the redundancy in the genetic code, for example, or variants that result in different amino acid sequence(s) but encode a protein or polypeptide having the same function), or variants having at least about 80%, at least about 81%, at least about 82%, at least about 83%, at least about 84%, at least about 85%, at least about 86%, at least about 87, at least about 88%, at least about 89%, at least about 90%, at least about 91%, at least about 92%, at least about 93%, at least about 94%, at least about 95%, at least about 96%, at least about 97%, at least about 98%, at least about 99% sequence identity to any one of the polypeptides encoded by SEQ ID NOs. 1-11.

The inventors discovered that a reverse genetics system can be used to generate RIX4414 strain rotavirus expressing heterologous polynucleotide sequences by fusing the heterologous polynucleotide sequences to the sequence encoding the rotaviral NSP3 protein. Thus, the inventors disclose herein that, in some embodiments, the disclosed compositions comprise a polynucleotide comprising a sequence encoding NSP3, e.g., (SEQ ID NO: 9), and further comprises a heterologous polynucleotide sequence.

The heterologous polynucleotide may encode a norovirus protein or peptide or a SARS-CoV-2 protein or peptide. The heterologous polynucleotide may encode NoV GII.4 MDA-145 VP1, NoV GII.4 MDA-145 P, NoV GII.4 MDA-145 P2, NoV GII.4 Cincinnati (Cin) VP1, NoV GII.4 Cincinnati (Cin) P, NoV GII.4 Sydney (Syd) VP1, NoV GII.4 Sydney (Syd) P, SARS-CoV-2 S1 portion of spike (S) protein, SARS-CoV-2 RBD region of S1 protein, SARS-CoV-2 Extended RBD region of S1 protein, SARS-CoV-2 glycosylated S1 portion of spike (S) protein including C-terminal CTMI domain of S2 portion of spike (S) protein, which may be encoded by SEQ ID NOs: 16-26, respectively, or a sequence with 85% similarity, 86% similarity, 87% similarity, 88% similarity, 89% similarity, 90% similarity, 91% similarity, 92% similarity, 93% similarity, 94% similarity, 95% similarity, 96% similarity, 97% similarity, 98% similarity, or 99% similarity to one of SEQ ID NOs: 16-26 (see Table 1 for full sequences).

In addition, Philip A A, Patton J T. 2022. Generation of Recombinant Rotaviruses Expressing Human Norovirus Capsid Proteins. Journal of Virology 96: No 22, published Oct. 31, 2022, which is incorporated herein by reference in its entirety, demonstrates that the inventors have successfully expressed norovirus (NoV) capsid protein as a heterologous polynucleotide in the RIX4414 reverse genetics system disclosed herein, see, for example, FIG. 5 of Phillip and Patton, 2022.

TABLE 1
Exemplary heterologous polynucleotides.

| SEQ ID | | |
|---|---|---|
| NO. | Description | Sequence |
| 16 | NoV GII.4 | ATGAAGATGGCGTCGAGTGACGCCAACCCATCTGATGG |
| | MDA-145 | GTCCGCAGCCAACCTCGTCCCAGAGGTCAACAATGAGGT |
| | VP1 | TATGGCTCTGGAGCCCGTTGTTGGTGCCGCTATTGCGGC |
| | | ACCTGTAGCGGGCCAACAAAATATAATTGACCCCTGGAT |
| | | TAGAAATAATTTTGTACAAGCCCCTGGTGGAGAGTTTAC |
| | | AGTGTCCCCTAGAAACGCTCCAGGTGAGATACTATGGAG |
| | | CGCGCCCTTGGGCCCTGATTTGAACCCCTATCTTTCTCAT |
| | | TTGTCCAGAATGTACAATGGTTATGCAGGCGGTTTCGAA |
| | | GTGCAAGTAATCCTCGCGGGGAACGCGTTCACCGCCGGG |
| | | AAAGTTATATTTGCAGCAGTTCCACCAAACTTTCCAACT |
| | | GAAGGCTTAAGCCCCAGCCAGGTTACTATGTTCCCCCAT |
| | | ATAATTGTAGATGTTAGGCAATTGGAACCTGTGTTGATC |
| | | CCCCTACCTGATGTTAGGAATAATTTCTATCATTACAATC |
| | | AATCACATGATTCTACCCTTAAGTTGATAGCAATGTTGT |
| | | ATACACCACTCAGGGCTAATAATGCCGGGGACGATGTCT |
| | | TCACAGTCTCTTGTCGAGTTCTCACGAGGCCATCCCCCG |
| | | ATTTTGATTTCATATTCTTGGTGCCACCCACAGTTGAATC |
| | | AAGAACTAAACCATTCACCGTCCCAATCTTAACTGTTGA |
| | | GGAAATGTCCAATTCAAGATTCCCCATTCCTTTGGAAAA |
| | | GTTGTACACGGGTCCTAGCAGTGCTTTTGTTGTCCAACC |
| | | ACAAAATGGCAGATGCACGACTGATGGCGTGCTCTTAGG |
| | | TACTACCCAGCTGTCAGCTGTCAACATCTGTAACTTTAG |
| | | GGGGGATGTCACCCATATTGTGGGCAGCCATGATTATAC |
| | | AATGAATCTGGCTTCCCAAAATTGGAGCAATTATGACCC |
| | | AACAGAAGAAATCCCAGCCCCCCTGGGAACACCAGATT |
| | | TTGTGGGGAAGATCCAAGGCCTGCTCACCCAGACCACAA |
| | | GAGCGGATGGCTCGACCCGTGCCCACAAAGCTACAGTG |
| | | AGCACTGGGAGTGTCCACTTCACTCCAAAGCTGGGTAGT |
| | | GTTCAATTCACCACTGACACAAACAATGATTTCCAAACT |
| | | GGCCAAAACACGAAATTCACCCCAGTTGGCGTCATCCAG |
| | | GACGGTGATCACCATCAGAATGAGCCCCAACAATGGGT |
| | | ACTCCCAAATTACTCAGGTAGAACTGGTCATAATGTGCA |
| | | CCTGGCCCCTGCCGTTGCCCCCACTTTTCCGGGTGAGCA |
| | | ACTCCTTTTCTTTAGATCCACTATGCCCGGATGTAGCGGG |
| | | TATCCCAACATGAATTTGGATTGCCTACTCCCCCAGGAA |
| | | TGGGTGCTGCACTTCTACCAGGAAGCAGCTCCAGCACAA |
| | | TCCGATGTGGCTCTGCTGAGATTTGTGAATCCAGACACA |
| | | GGTAGGGTTCTGTTTGAGTGCAAGCTCCATAAATCAGGC |
| | | TATATCACAGTGGCTCACACCGGCCCGTATGACTTGGTT |
| | | ATCCCCCCCAATGGTTATTTTAGATTTGATTCCTGGGTCA |
| | | ACCAGTTCTACACACTTGCCCCCATGGGAAATGGAACGG |
| | | GGCGCAGGCGTGCATTATAA |
| 17 | NoV GII.4 | AAACCATTCACCGTCCCAATCTTAACTGTTGAGGAAATG |
| | MDA-145P | TCCAATTCAAGATTCCCCATTCCTTTGGAAAAGTTGTAC |
| | | ACGGGTCCTAGCAGTGCTTTTGTTGTCCAACCACAAAAT |
| | | GGCAGATGCACGACTGATGGCGTGCTCTTAGGTACTACC |
| | | CAGCTGTCAGCTGTCAACATCTGTAACTTTAGGGGGGAT |
| | | GTCACCCATATTGTGGGCAGCCATGATTATACAATGAAT |
| | | CTGGCTTCCCAAAATTGGAGCAATTATGACCCAACAGAA |
| | | GAAATCCCAGCCCCCCTGGGAACACCAGATTTTGTGGGG |
| | | AAGATCCAAGGCCTGCTCACCCAGACCACAAGAGCGGA |
| | | TGGCTCGACCCGTGCCCACAAAGCTACAGTGAGCACTGG |
| | | GAGTGTCCACTTCACTCCAAAGCTGGGTAGTGTTCAATT |
| | | CACCACTGACACAAACAATGATTTCCAAACTGGCCAAAA |
| | | CACGAAATTCACCCCAGTTGGCGTCATCCAGGACGGTGA |
| | | TCACCATCAGAATGAGCCCCAACAATGGGTACTCCCAAA |
| | | TTACTCAGGTAGAACTGGTCATAATGTGCACCTGGCCCC |
| | | TGCCGTTGCCCCCACTTTTCCGGGTGAGCAACTCCTTTTC |
| | | TTTAGATCCACTATGCCCGGATGTAGCGGGTATCCCAAC |
| | | ATGAATTTGGATTGCCTACTCCCCCAGGAATGGGTGCTG |
| | | CACTTCTACCAGGAAGCAGCTCCAGCACAATCCGATGTG |
| | | GCTCTGCTGAGATTTGTGAATCCAGACACAGGTAGGGTT |
| | | CTGTTTGAGTGCAAGCTCCATAAATCAGGCTATATCACA |
| | | GTGGCTCACACCGGCCCGTATGACTTGGTTATCCCCCCC |
| | | AATGGTTATTTTAGATTTGATTCCTGGGTCAACCAGTTCT |
| | | ACACACTTGCCCCCATGGGAAATGGAACGGGGCGCAGG |
| | | CGTGCATTATAA |
| 18 | NoV GII.4 | ACTACCCAGCTGTCAGCTGTCAACATCTGTAACTTTAGG |
| | MDA-145 | GGGGATGTCACCCATATTGTGGGCAGCCATGATTATACA |
| | P2 | ATGAATCTGGCTTCCCAAAATTGGAGCAATTATGACCCA |
| | | ACAGAAGAAATCCCAGCCCCCCTGGGAACACCAGATTTT |
| | | GTGGGGAAGATCCAAGGCCTGCTCACCCAGACCACAAG |
| | | AGCGGATGGCTCGACCCGTGCCCACAAAGCTACAGTGA |
| | | GCACTGGGAGTGTCCACTTCACTCCAAAGCTGGGTAGTG |
| | | TTCAATTCACCACTGACACAAACAATGATTTCCAAACTG |
| | | GCCAAAACACGAAATTCACCCCAGTTGGCGTCATCCAGG |
| | | ACGGTGATCACCATCAGAATGAGCCCCAACAATGGGTA |
| | | CTCCCAAATTACTCAGGTAGAACTGGTCATAATGTGCAC |
| | | CTGTGA |
| 19 | NoV GII.4 | ATGAAGATGGCGTCTAAGGACGCCAACCCATCTGATGG |
| | Cincinnati | GTCCACAGCCAACCTCGTCCCAGAGGTCAACAATGAGGT |
| | (Cin) VP1 | TATGGCTTTGGAGCCCGTTGTTGGTGCCGCTATTGCGGC |
| | | ACCTGTAGCGGGCCAACAAAATGTAATTGACCCCTGGAT |
| | | TAGAAATAATTTTGTACAAGCCCCTGGTGGAGAGTTTAC |
| | | AGTATCCCCTAGAAACGCTCCAGGTGAAATACTATGGAG |
| | | CGCGCCCTTGGGCCCCGATCTGAATCCCTACCTTTCTCAT |
| | | TTGGCCAGAATGTACAATGGCTATGCAGGTGGCTTTGAA |
| | | GTGCAGGTAATCCTTGCGGGGAATGCGTTCACCGCCGGG |
| | | AAAGTCATATTTGCAGCAGTCCCACCAAATTTCCCAACT |
| | | GAAGGCTTGAGCCCCAGCCAGGTCACTATGTTCCCCCAT |
| | | ATAGTAGTAGATGTTAGGCAACTGGAACCTGTGTTGATC |
| | | CCCTTACCTGATGTTAGGAACAATTTCTATCACTACAAC |
| | | CAATCAAATGACCCCACCATTAAACTGATAGCAATGCTG |
| | | TACACACCACTTAGGGCTAATAATGCTGGGGAAGATGTC |
| | | TTCACAGTCTCTTGTCGAGTCCTCACGAGGCCATCCCCC |
| | | GATTTTGATTTCATATTTTTGGTGCCACCCACAGTTGAGT |
| | | CAAGAACTAAACCATTCACCGTCCCAATTTTAACTGTTG |
| | | AGGAAATGACCAATTCAAGATTCCCCATTCCTTTGGAAA |
| | | AGTTGTTCACGGGTCCTAGCGGTGCCTTTGTTGTTCAACC |
| | | ACAAAATGGCAGGTGCACGACTGATGGCGTGCTCTTAGG |
| | | CACTACCCAACTGTCTCCTGTCAACATCTGCACCTTCAG |
| | | AGGGGATGTCACCCACATTGCAGGTACTCATAATTACAC |
| | | AATGAATTTGGCTTCTCAAAATTGGAACAATTATGACCC |
| | | AACAGAAGAAATCCCAGCCCCTCTGGGGACCCCAGATTT |
| | | CGTGGGAAGGATCCAAGGCATGCTCACCCAAACTACAA |
| | | GGGGAGATGGCTCTACCCGCGGTCACAAAGCTACAGTG |
| | | AGCACTGGGGATGTCCACTTCACTCCAAAGCTGGGCAGT |
| | | ATTCAGTTCAACACTGATACAAACAATGATTTTGAAACT |
| | | GGCCAAAACACGAAATTCACCCCAGTCGGTGTTGTCCAG |
| | | GATGGTAATGGTACCCACCAAAATGAACCTCAACAATG |
| | | GGTGCTCCCAAGCTACTCAGGTAGAACTGGTCATAATGT |
| | | ACACCTAGCCCCCGCCGTAGCCCCCACTTTTCCGGGTGA |
| | | GCAACTTCTTTTCTTCAGGTCCACTATGCCCGGATGCAGC |
| | | GGGTACCCTAACATGAATTTGGATTGCCTACTCCCCCAA |
| | | GAATGGGTGCAGCACTTCTACCAAGAGGCAGCTCCAGC |
| | | ACAATCTGATGTGGCTCTGCTAAGATTTGTGAATCCAGA |
| | | CACAGGTAGGGTTCTATTTGAGTGCAAGCTTCATAAATC |
| | | AGGCTATGTCACAGTGGCTCACACTGGCCAGCATGATTT |
| | | GGTCATCCCCCCCAATGGCTACTTTAGATTTGATTCCTGG |
| | | GTCAACCAATTCTACACACTTGCCCCCCTGGGAAATGGG |
| | | ACGGGGCGTAGACGTGCATTATAA |
| 20 | NoV GII.4 | AAACCATTCACCGTCCCAATTTTAACTGTTGAGGAAATG |
| | Cincinnati | ACCAATTCAAGATTCCCCATTCCTTTGGAAAAGTTGTTC |
| | (Cin) P | ACGGGTCCTAGCGGTGCCTTTGTTGTTCAACCACAAAAT |
| | | GGCAGGTGCACGACTGATGGCGTGCTCTTAGGCACTACC |
| | | CAACTGTCTCCTGTCAACATCTGCACCTTCAGAGGGGAT |
| | | GTCACCCACATTGCAGGTACTCATAATTACACAATGAAT |
| | | TTGGCTTCTCAAAATTGGAACAATTATGACCCAACAGAA |
| | | GAAATCCCAGCCCCTCTGGGGACCCCAGATTTCGTGGGA |
| | | AGGATCCAAGGCATGCTCACCCAAACTACAAGGGGAGA |
| | | TGGCTCTACCCGCGGTCACAAAGCTACAGTGAGCACTGG |
| | | GGATGTCCACTTCACTCCAAAGCTGGGCAGTATTCAGTT |
| | | CAACACTGATACAAACAATGATTTTGAAACTGGCCAAAA |
| | | CACGAAATTCACCCCAGTCGGTGTTGTCCAGGATGGTAA |
| | | TGGTACCCACCAAAATGAACCTCAACAATGGGTGCTCCC |
| | | AAGCTACTCAGGTAGAACTGGTCATAATGTACACCTAGC |
| | | CCCCGCCGTAGCCCCCACTTTTCCGGGTGAGCAACTTCTT |
| | | TTCTTCAGGTCCACTATGCCCGGATGCAGCGGGTACCCT |
| | | AACATGAATTTGGATTGCCTACTCCCCCAAGAATGGGTG |
| | | CAGCACTTCTACCAAGAGGCAGCTCCAGCACAATCTGAT |
| | | GTGGCTCTGCTAAGATTTGTGAATCCAGACACAGGTAGG |
| | | GTTCTATTTGAGTGCAAGCTTCATAAATCAGGCTATGTC |
| | | ACAGTGGCTCACACTGGCCAGCATGATTTGGTCATCCCC |
| | | CCCAATGGCTACTTTAGATTTGATTCCTGGGTCAACCAA |
| | | TTCTACACACTTGCCCCCCTGGGAAATGGGACGGGGCGT |
| | | AGACGTGCATTATAA |
| 21 | NoV GII.4 | ATGAAGATGGCGTCGAGTGACGCCAACCCATCTGATGG |
| | Sydney | GTCCGCAGCCAACCTCGTCCCAGAGGTCAACAATGAGGT |
| | (Syd) VP1 | TATGGCTCTGGAGCCCGTTGTTGGTGCCGCCATTGCGGC |
| | | ACCTGTAGCGGGCCAACAAAATGTAATTGACCCCTGGAT |
| | | TAGAAATAATTTTGTACAAGCCCCTGGTGGAGAGTTTAC |
| | | AGTGTCCCCTAGAAACGCTCCAGGTGAAATACTATGGAG |
| | | CGCGCCCTTGGGCCCTGATCTAAATCCCTACCTATCCCAT |
| | | TTGGCCAGAATGTACAATGGTTATGCAGGTGGTTTTGAA |
| | | GTGCAGGTAATTCTCGCGGGGAACGCGTTCACCGCCGGG |
| | | AAGGTCATATTTGCAGCAGTCCCACCAAATTTTCCAACT |
| | | GAAGGCTTGAGCCCCAGCCAGGTCACTATGTTCCCCCAT |
| | | ATAGTAGTAGATGTTAGGCAATTAGAACCTGTGTTGATT |
| | | CCCTTACCCGATGTTAGGAATAATTTCTATCATTACAATC |
| | | AATCAAATGACCCCACCATTAAGTTGATAGCAATGTTGT |
| | | ATACACCACTTAGGGCTAATAATGCTGGGGATGATGTCT |
| | | TCACAGTTTCTTGCCGAGTTCTCACGAGACCATCCCCCG |
| | | ATTTTGATTTCATATTTCTAGTACCACCCACAGTTGAGTC |
| | | AAGAACTAAACCATTCTCTGTCCCAGTTTTAACTGTTGA |
| | | GGAGATGACCAATTCAAGATTCCCCATTCCTTTGGAAAA |
| | | GTTGTTCACGGGTCCCAGCAGTGCCTTTGTTGTCCAACC |
| | | ACAAAACGGTAGGTGCACGACTGATGGCGTGCTCCTAG |
| | | GCACCACCCAACTGTCTCCTGTCAACATCTGCACCTTCA |
| | | GAGGAGATGTCACCCATATCACAGGTAGTCGTAACTACA |
| | | CAATGAATTTGGCTTCTCAAAATTGGAGCAATTATGACC |
| | | CAACAGAAGAAATCCCAGCCCCTCTAGGAACTCCAGATT |
| | | TTGTGGGGAAGATTCAAGGCGTGCTCACCCAAACCACAA |
| | | GGACAGATGGCTCAACACGCGGCCACAAAGCCACAGTG |
| | | TACACTGGGAGCGCCGACTTTGCTCCAAAACTGGGTAGA |
| | | GTTCAATTTGGAACTGACACAGACCATGATTTTGAAGCT |
| | | AACCAAAACACAAAATTCACCCCAGTTGGTGTCATCCAA |
| | | GATGGTAGCACCACCCACCGAAATGAACCCCAACAGTG |
| | | GGTGCTCCCAAGTTACTCAGGCAGAAATACTCCTAATGT |
| | | GCATCTGGCCCCCGCTGTAGCCCCCACTTTTCCGGGTGA |
| | | GCAACTTCTCTTCTTCAGATCCACCATGCCCGGATGCAG |
| | | CGGGTACCCCAACATGGATTTGGACTGTCTGCTCCCCCA |
| | | GGAATGGGTGCAGTACTTCTACCAAGAGGCAGCCCCAG |
| | | CACAATCTGATGTGGCTCTGCTAAGATTTGTGAATCCAG |
| | | ACACAGGTAGGGTTTTGTTTGAGTGTAAGCTTCATAAAT |
| | | CAGGCTATGTTACAGTGGCTCACACTGGCCAACATGATT |
| | | TGGTTATCCCCCCCAATGGTTATTTTAGGTTTGATTCCTG |
| | | GGTCAACCAGTTTTACACGCTTGCCCCCATGGGAAATGG |
| | | AACGGGGCGTAGACGTGCACTATAA |
| 22 | NoV GII.4 | AAACCATTCTCTGTCCCAGTTTTAACTGTTGAGGAGATGACCA |
| | Sydney | ATTCAAGATTCCCCATTCCTTTGGAAAAGTTGTTCACGGGTCC |
| | (Syd) P | CAGCAGTGCCTTTGTTGTCCAACCACAAAACGGTAGGTGCAC |
| | | GACTGATGGCGTGCTCCTAGGCACCACCCAACTGTCTCCTGTC |
| | | AACATCTGCACCTTCAGAGGAGATGTCACCCATATCACAGGT |
| | | AGTCGTAACTACACAATGAATTTGGCTTCTCAAAATTGGAGCA |
| | | ATTATGACCCAACAGAAGAAATCCCAGCCCCTCTAGGAACTC |
| | | CAGATTTTGTGGGGAAGATTCAAGGCGTGCTCACCCAAACCA |
| | | CAAGGACAGATGGCTCAACACGCGGCCACAAAGCCACAGTGT |
| | | ACACTGGGAGCGCCGACTTTGCTCCAAAACTGGGTAGAGTTC |
| | | AATTTGGAACTGACACAGACCATGATTTTGAAGCTAACCAAA |
| | | ACACAAAATTCACCCCAGTTGGTGTCATCCAAGATGGTAGCA |
| | | CCACCCACCGAAATGAACCCCAACAGTGGGTGCTCCCAAGTT |
| | | ACTCAGGCAGAAATACTCCTAATGTGCATCTGGCCCCCGCTGT |
| | | AGCCCCCACTTTTCCGGGTGAGCAACTTCTCTTCTTCAGATCC |
| | | ACCATGCCCGGATGCAGCGGGTACCCCAACATGGATTTGGAC |
| | | TGTCTGCTCCCCCAGGAATGGGTGCAGTACTTCTACCAAGAGG |
| | | CAGCCCCAGCACAATCTGATGTGGCTCTGCTAAGATTTGTGAA |
| | | TCCAGACACAGGTAGGGTTTTGTTTGAGTGTAAGCTTCATAAA |
| | | TCAGGCTATGTTACAGTGGCTCACACTGGCCAACATGATTTGG |
| | | TTATCCCCCCCAATGGTTATTTTAGGTTTGATTCCTGGGTCAAC |
| | | CAGTTTTACACGCTTGCCCCCATGGGAAATGGAACGGGGCGT |
| | | AGACGTGCACTATAA |
| 23 | SARS- | GTGTTTGTTTTTCTTGTTTTATTGCCACTAGTCTCTAGTCA |
| | CoV-2 S1 | GTGTGTTAATCTTACAACCAGAACTCAATTACCCCCTGC |
| | portion of | ATACACTAATTCTTTCACACGTGGTGTTTATTACCCTGAC |
| | spike (S) | AAAGTTTTCAGATCCTCAGTTTTACATTCAACTCAGGACT |
| | protein | TGTTCTTACCTTTCTTTTCCAATGTTACTTGGTTCCATGCT |
| | | ATACATGTCTCTGGGACCAATGGTACTAAGAGGTTTGAT |
| | | AACCCTGTCCTACCATTTAATGATGGTGTTTATTTTGCTT |
| | | CCACTGAGAAGTCTAACATAATAAGAGGCTGGATTTTTG |
| | | GTACTACTTTAGATTCGAAGACCCAGTCCCTACTTATTGT |
| | | TAATAACGCTACTAATGTTGTTATTAAAGTCTGTGAATTT |
| | | CAATTTTGTAATGATCCATTTTTGGGTGTTTATTACCACA |
| | | AAAACAACAAAAGTTGGATGGAAAGTGAGTTCAGAGTT |
| | | TATTCTAGTGCGAATAATTGCACTTTTGAATATGTCTCTC |
| | | AGCCTTTTCTTATGGACCTTGAAGGAAAACAGGGTAATT |
| | | TCAAAAATCTTAGGGAATTTGTGTTTAAGAATATTGATG |
| | | GTTATTTTAAAATATATTCTAAGCACACGCCTATTAATTT |
| | | AGTGCGTGATCTCCCTCAGGGTTTTTCGGCTTTAGAACC |
| | | ATTGGTAGATTTGCCAATAGGTATTAACATCACTAGGTT |
| | | TCAAACTTTACTTGCTTTACATAGAAGTTATTTGACTCCT |
| | | GGTGATTCTTCTTCAGGTTGGACAGCTGGTGCTGCAGCT |
| | | TATTATGTGGGTTATCTTCAACCTAGGACTTTTCTATTAA |
| | | AATATAATGAAAATGGAACCATTACAGATGCTGTAGACT |
| | | GTGCACTTGACCCTCTCTCAGAAACAAAGTGTACGTTGA |
| | | AATCCTTCACTGTAGAAAAAGGAATCTATCAAACTTCTA |
| | | ACTTTAGAGTCCAACCAACAGAATCTATTGTTAGATTTC |
| | | CTAATATTACAAACTTGTGCCCTTTTGGTGAAGTTTTTAA |
| | | CGCCACCAGATTTGCATCTGTTTATGCTTGGAACAGGAA |
| | | GAGAATCAGCAACTGTGTTGCTGATTATTCTGTCCTATAT |
| | | AATTCCGCATCATTTTCCACTTTTAAGTGTTATGGAGTGT |
| | | CTCCTACTAAATTAAATGATCTCTGCTTTACTAATGTCTA |
| | | TGCAGATTCATTTGTAATTAGAGGTGATGAAGTCAGACA |
| | | AATCGCTCCAGGGCAAACTGGAAAGATTGCTGATTATAA |
| | | TTATAAATTACCAGATGATTTTACAGGCTGCGTTATAGC |
| | | TTGGAATTCTAACAATCTTGATTCTAAGGTTGGTGGTAA |
| | | TTATAATTACCTGTATAGATTGTTTAGGAAGTCTAATCTC |
| | | AAACCTTTTGAGAGAGATATTTCAACTGAAATCTATCAG |
| | | GCCGGTAGCACACCTTGTAATGGTGTTGAAGGTTTTAAT |
| | | TGTTACTTTCCTTTACAATCATATGGTTTCCAACCCACTA |
| | | ATGGTGTTGGTTACCAACCATACAGAGTAGTAGTACTTT |
| | | CTTTTGAACTTCTACATGCACCAGCAACTGTTTGTGGACC |
| | | TAAAAAGTCTACTAATTTGGTTAAAAACAAATGTGTCAA |
| | | TTTCAACTTCAATGGTTTAACAGGCACAGGTGTTCTTACT |
| | | GAGTCTAACAAAAAGTTTCTGCCTTTCCAACAATTTGGC |
| | | AGAGACATTGCTGACACTACTGATGCTGTCCGTGATCCA |
| | | CAGACACTTGAGATTCTTGACATTACACCATGTTCTTTTG |
| | | GTGGTGTCAGTGTTATAACACCAGGAACAAATACTTCTA |
| | | ACCAGGTTGCTGTTCTTTATCAGGATGTTAACTGCACAG |
| | | AAGTCCCTGTTGCTATTCATGCAGATCAACTTACTCCTAC |
| | | TTGGCGTGTTTATTCTACAGGTTCTAATGTTTTTCAAACA |
| | | CGTGCAGGCTGTTTAATAGGGGCTGAACATGTCAACAAC |
| | | TCATATGAGTGTGACATACCCATTGGTGCAGGTATATGC |
| | | GCTAGTTATCAGACTCAGACTAATTCTCCTCGGCGGGCA |
| | | CGT |
| 24 | SARS- | TTGTGCCCTTTTGGTGAAGTTTTTAACGCCACCAGATTTG |
| | CoV-2 | CATCTGTTTATGCTTGGAACAGGAAGAGAATCAGCAACT |
| | RBD | GTGTTGCTGATTATTCTGTCCTATATAATTCCGCATCATT |
| | region of | TTCCACTTTTAAGTGTTATGGAGTGTCTCCTACTAAATTA |
| | S1 protein | AATGATCTCTGCTTTACTAATGTCTATGCAGATTCATTTG |
| | | TAATTAGAGGTGATGAAGTCAGACAAATCGCTCCAGGG |
| | | CAAACTGGAAAGATTGCTGATTATAATTATAAATTACCA |
| | | GATGATTTTACAGGCTGCGTTATAGCTTGGAATTCTAAC |
| | | AATCTTGATTCTAAGGTTGGTGGTAATTATAATTACCTGT |
| | | ATAGATTGTTTAGGAAGTCTAATCTCAAACCTTTTGAGA |
| | | GAGATATTTCAACTGAAATCTATCAGGCCGGTAGCACAC |
| | | CTTGTAATGGTGTTGAAGGTTTTAATTGTTACTTTCCTTT |
| | | ACAATCATATGGTTTCCAACCCACTAATGGTGTTGGTTA |
| | | CCAACCATACAGAGTAGTAGTACTTTCTTTTGAACTTCTA |
| | | CATGCACCAGCAACT |
| 25 | SARS- | GGAATCTATCAAACTTCTAACTTTAGAGTCCAACCAACA |
| | CoV-2 | GAATCTATTGTTAGATTTCCTAATATTACAAACTTGTGCC |
| | Extended | CTTTTGGTGAAGTTTTTAACGCCACCAGATTTGCATCTGT |
| | RBD | TTATGCTTGGAACAGGAAGAGAATCAGCAACTGTGTTGC |
| | region of | TGATTATTCTGTCCTATATAATTCCGCATCATTTTCCACT |
| | S1 protein | TTTAAGTGTTATGGAGTGTCTCCTACTAAATTAAATGATC |
| | | TCTGCTTTACTAATGTCTATGCAGATTCATTTGTAATTAG |
| | | AGGTGATGAAGTCAGACAAATCGCTCCAGGGCAAACTG |
| | | GAAAGATTGCTGATTATAATTATAAATTACCAGATGATT |
| | | TTACAGGCTGCGTTATAGCTTGGAATTCTAACAATCTTG |
| | | ATTCTAAGGTTGGTGGTAATTATAATTACCTGTATAGATT |
| | | GTTTAGGAAGTCTAATCTCAAACCTTTTGAGAGAGATAT |
| | | TTCAACTGAAATCTATCAGGCCGGTAGCACACCTTGTAA |
| | | TGGTGTTGAAGGTTTTAATTGTTACTTTCCTTTACAATCA |
| | | TATGGTTTCCAACCCACTAATGGTGTTGGTTACCAACCA |
| | | TACAGAGTAGTAGTACTTTCTTTTGAACTTCTACATGCAC |
| | | CAGCAACTGTTTGTGGACCTAAAAAGTCTACTAATTTGG |
| | | TTAAAAACAAATGTGTCAATTTCAACTTCAATGGTTTAA |
| | | CAGGCACAGGTGTTCTTACTGAGTCTAACAAAAAGTTTC |
| | | TGCCTTTCCAACAATTTGGCAGAGACATTGCTGACACTA |
| | | CTGATGCTGTCCGTGATCCACAGACACTTGAGATTCTTG |
| | | ACATTACACCATGTTCTTTTGGTGGTGTCAGTGTTATAAC |
| | | A |
| 26 | SARS- | GTGTTTGTTTTTCTTGTTTTATTGCCACTAGTCTCTAGTCA |
| | CoV-2 | GTGTGTTAATCTTACAACCAGAACTCAATTACCCCCTGC |
| | glycosylated | ATACACTAATTCTTTCACACGTGGTGTTTATTACCCTGAC |
| | S1 | AAAGTTTTCAGATCCTCAGTTTTACATTCAACTCAGGACT |
| | portion of | TGTTCTTACCTTTCTTTTCCAATGTTACTTGGTTCCATGCT |
| | spike (S) | ATACATGTCTCTGGGACCAATGGTACTAAGAGGTTTGAT |
| | protein, | AACCCTGTCCTACCATTTAATGATGGTGTTTATTTTGCTT |
| | includes C- | CCACTGAGAAGTCTAACATAATAAGAGGCTGGATTTTTG |
| | terminal | GTACTACTTTAGATTCGAAGACCCAGTCCCTACTTATTGT |
| | CTMI | TAATAACGCTACTAATGTTGTTATTAAAGTCTGTGAATTT |
| | domain of | CAATTTTGTAATGATCCATTTTTGGGTGTTTATTACCACA |
| | S2 portion | AAAACAACAAAAGTTGGATGGAAAGTGAGTTCAGAGTT |
| | of spike (S) | TATTCTAGTGCGAATAATTGCACTTTTGAATATGTCTCTC |
| | protein | AGCCTTTTCTTATGGACCTTGAAGGAAAACAGGGTAATT |
| | | TCAAAAATCTTAGGGAATTTGTGTTTAAGAATATTGATG |
| | | GTTATTTTAAAATATATTCTAAGCACACGCCTATTAATTT |
| | | AGTGCGTGATCTCCCTCAGGGTTTTTCGGCTTTAGAACC |
| | | ATTGGTAGATTTGCCAATAGGTATTAACATCACTAGGTT |
| | | TCAAACTTTACTTGCTTTACATAGAAGTTATTTGACTCCT |
| | | GGTGATTCTTCTTCAGGTTGGACAGCTGGTGCTGCAGCT |
| | | TATTATGTGGGTTATCTTCAACCTAGGACTTTTCTATTAA |
| | | AATATAATGAAAATGGAACCATTACAGATGCTGTAGACT |
| | | GTGCACTTGACCCTCTCTCAGAAACAAAGTGTACGTTGA |
| | | AATCCTTCACTGTAGAAAAAGGAATCTATCAAACTTCTA |
| | | ACTTTAGAGTCCAACCAACAGAATCTATTGTTAGATTTC |
| | | CTAATATTACAAACTTGTGCCCTTTTGGTGAAGTTTTTAA |
| | | CGCCACCAGATTTGCATCTGTTTATGCTTGGAACAGGAA |
| | | GAGAATCAGCAACTGTGTTGCTGATTATTCTGTCCTATAT |
| | | AATTCCGCATCATTTTCCACTTTTAAGTGTTATGGAGTGT |
| | | CTCCTACTAAATTAAATGATCTCTGCTTTACTAATGTCTA |
| | | TGCAGATTCATTTGTAATTAGAGGTGATGAAGTCAGACA |
| | | AATCGCTCCAGGGCAAACTGGAAAGATTGCTGATTATAA |
| | | TTATAAATTACCAGATGATTTTACAGGCTGCGTTATAGC |
| | | TTGGAATTCTAACAATCTTGATTCTAAGGTTGGTGGTAA |
| | | TTATAATTACCTGTATAGATTGTTTAGGAAGTCTAATCTC |
| | | AAACCTTTTGAGAGAGATATTTCAACTGAAATCTATCAG |
| | | GCCGGTAGCACACCTTGTAATGGTGTTGAAGGTTTTAAT |
| | | TGTTACTTTCCTTTACAATCATATGGTTTCCAACCCACTA |
| | | ATGGTGTTGGTTACCAACCATACAGAGTAGTAGTACTTT |
| | | CTTTTGAACTTCTACATGCACCAGCAACTGTTTGTGGACC |
| | | TAAAAAGTCTACTAATTTGGTTAAAAACAAATGTGTCAA |
| | | TTTCAACTTCAATGGTTTAACAGGCACAGGTGTTCTTACT |
| | | GAGTCTAACAAAAAGTTTCTGCCTTTCCAACAATTTGGC |
| | | AGAGACATTGCTGACACTACTGATGCTGTCCGTGATCCA |
| | | CAGACACTTGAGATTCTTGACATTACACCATGTTCTTTTG |
| | | GTGGTGTCAGTGTTATAACACCATGGTACATTTGGCTAG |
| | | GTTTTATAGCTGGCTTGATTGCCATAGTAATGGTGACAA |
| | | TTATGCTTTGCTGTATGACCAGTTGCTGTAGTTGTCTCAA |
| | | GGGCTGTTGTTCTTGTGGATCCTGCTGCAAATTTGATGA |
| | | AGACGACTCTGAGCCAGTGCTCAAAGGAGTCAAATTAC |
| | | ATTACACA |

In some embodiments, the heterologous polynucleotide encodes a protein or peptide. In some embodiments, the sequence encoding NSP3, e.g., SEQ ID NO: 9, further comprises the heterologous polynucleotide fused to the 3′ end of the sequence such that the heterologous polynucleotide encodes a protein or peptide in frame with the NSP3 sequence, thereby allowing transcription of a single mRNA that encodes both NSP3 and the heterologous polynucleotide. In some embodiments, the polynucleotide comprising a sequence encoding NSP3 and a heterologous polynucleotide comprise a sequence encoding a cleavage site. In some embodiments, the cleavage site is a self-cleaving peptide, e.g., porcine teschovirus P2A element (SEQ ID NO: 13). Thus, in some embodiments, the disclosed compositions comprise, from 5′ to 3′, a polynucleotide encoding NSP3, fused in-frame to a sequence encoding a self-cleaving peptide which is fused in-frame to a heterologous polynucleotide sequence encoding a peptide or protein. Accordingly, transcription and translation of such compositions results in production of a fusion protein comprising, from N- to C-terminus, a rotavirus NSP3 protein fused to a self-cleaving peptide, e.g., SEQ ID NO: 13, which is fused to a peptide or protein encoded by the heterologous polynucleotide, in a cell; following translation, the fusion protein self-cleaves resulting in two separate proteins (1) a functional rotavirus NSP3 protein and (2) the protein or peptide encoded by the heterologous polynucleotide. In some embodiments, the compositions further comprise a sequence encoding a sequence encoding a linker, e.g., a flexible linker located 3′ to, and in frame with, the sequence encoding NSP3 protein and 5′ to a cleavage site. Without being limited by any theory or mechanism, the inventors believe that the addition of a flexible linker between the NSP3 protein and the cleavage site improves cleavage. In some embodiments, the linker is a (GAG)ₙ linker (also referred to as a GAG linker), wherein n=1, 2, 3, 4, 5, 6, 7, 8, 9, 10, or more, or a (GSG)ₙ linker (also referred to as a GSG linker), wherein n=1, 2, 3, 4, 5, 6, 7, 8, 9, 10 or more.

In some embodiments, the sequence encoding a cleavage site encodes a protease cleavage site, e.g., a thrombin cleavage site, e.g., SEQ ID NO: 12.

The inventors further contemplate that the heterologous polynucleotide sequence described above comprise sequences encoding proteins or peptides derived from infectious organisms, e.g., norovirus or SARS-CoV-2. Therefore, in some embodiments, the disclosed compositions comprise sequences encoding rotavirus NSP3 fused, in-frame, to a heterologous polynucleotide encoding a norovirus protein or peptide, e.g., norovirus VP1, or SARS-CoV-2 protein or peptide, e.g., SARS-CoV-2 surface glycoprotein.

In some embodiments, the compositions comprise a polynucleotide comprising a sequence encoding a recombinant rotavirus NSP3, e.g., SEQ ID NO:9, wherein the polynucleotide encodes a positive sense viral transcript, wherein the polynucleotide further comprises a heterologous polynucleotide in frame with the sequence encoding a recombinant rotavirus NSP3, wherein the heterologous polynucleotide encodes a peptide or protein comprising a norovirus or SARS-CoV-2 peptide or protein, or a fragment thereof. In some embodiments, the compositions further comprise a sequence encoding a self-cleaving peptide, e.g., a sequence encoding SEQ ID NO: 13, fused, in-frame, between the polynucleotide and the heterologous polynucleotide. Therefore, transcription and translation of said composition results in, from N- to C-terminus, a functional rotavirus NSP3 protein, self-cleaving linker, e.g., SEQ ID NO: 13, and a norovirus protein or peptide, e.g., norovirus VP1, or SARS-CoV-2 protein or peptide, or a fragment thereof.

### Infectious Particles

In another aspect of the current disclosure, infectious particles are provided. In some embodiments, the infectious particles comprise a composition comprising a polynucleotide comprising a sequence encoding a recombinant rotavirus protein, wherein the polynucleotide encodes a positive sense viral transcript.

As used herein, “infectious particles” refers to any particle capable of causing an infection of an organism or cell. Exemplary infectious particles include, but are not limited to, viral particles, or virions and the like. The terms “virus,” “viral particle,” and “virion” are used interchangeably herein.

Without wishing to be limited, the instant disclosure provides compositions comprising polynucleotides encoding rotavirus proteins operably linked to a promoter, e.g., a T7 promoter (SEQ ID NO: 13). In some embodiments, the compositions may be used in a reverse genetics approach to generate recombinant rotavirus, e.g., recombinant rotavirus strain RIX4414. Thus, some embodiments, the recombinant rotavirus may comprise the disclosed compositions.

The disclosed infectious particles, e.g., recombinant rotaviruses, may comprise one or more heterologous proteins or peptides, e.g., norovirus or SARS-CoV-2 proteins or peptides, as described above. The heterologous proteins or peptides may be encoded in the infectious particle, e.g., viral, genome and subsequently produced during viral replication. Such infectious particles comprising a heterologous protein or peptide may be advantageous in eliciting an immune response in a subject or as a vaccine composition.

### Pharmaceutical Compositions

The inventors disclose herein compositions, methods, and systems useful in making recombinant rotavirus that may be suitable for administration to subjects. Therefore, in another aspect of the current disclosure, pharmaceutical compositions are provided. In some embodiments, the pharmaceutical compositions comprise an infectious particle comprising a composition comprising a polynucleotide comprising a sequence encoding a recombinant rotavirus protein, wherein the polynucleotide encodes a positive sense viral transcript. In some embodiments, the pharmaceutical compositions comprise an infectious particle made by transfecting a cell with a composition comprising a polynucleotide comprising a sequence encoding a recombinant rotavirus protein, wherein the polynucleotide encodes a positive sense viral transcript.

The disclosed compositions and methods may be administered as pharmaceutical compositions and, therefore, pharmaceutical compositions incorporating the compounds are considered to be embodiments of the disclosed compositions. Such compositions may take any physical form which is pharmaceutically acceptable; illustratively, they can be orally administered pharmaceutical compositions. Such pharmaceutical compositions contain an effective amount of a disclosed composition, which effective amount is related to the daily dose of the composition to be administered. Each dosage unit may contain the daily dose of a given composition or each dosage unit may contain a fraction of the daily dose, such as one-half or one-third of the dose. The amount of each composition to be contained in each dosage unit can depend, in part, on the identity of the particular composition chosen for the therapy and other factors, such as the indication for which it is given. The disclosed pharmaceutical compositions may be formulated to provide quick, sustained, or delayed release of the active ingredient after administration to the patient by employing well known procedures.

The pharmaceutical compositions may be utilized in methods of eliciting an immune response or vaccinating against a pathogen, e.g., rotavirus, norovirus, SARS-CoV-2. As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration. By way of example, a subject may be at risk for infection by a pathogen, e.g., rotavirus, norovirus, SARS-CoV-2, and administration of the disclosed pharmaceutical compositions elicits a protective immune response or vaccinates against the pathogen.

As used herein the term “effective amount” refers to the amount or dose of the compound, upon single or multiple dose administration to the subject, which provides the desired effect in the subject under diagnosis or treatment. The disclosed methods may include administering an effective amount of the disclosed compounds (e.g., as present in a pharmaceutical composition) for eliciting an immune response to a pathogen, e.g., rotavirus, norovirus, SARS-CoV-2, or vaccinating against the pathogen.

An effective amount can be readily determined by the attending diagnostician, as one skilled in the art, by the use of known techniques and by observing results obtained under analogous circumstances. In determining the effective amount or dose of composition administered, a number of factors can be considered by the attending diagnostician, such as: the species of the subject; its size, age, and general health; the degree of involvement or the severity of the disease or disorder involved; the response of the individual subject; the particular composition administered; the mode of administration; the bioavailability characteristics of the preparation administered; the dose regimen selected; the use of concomitant medication; and other relevant circumstances.

Oral administration is an illustrative route of administering the compositions and methods disclosed herein. Other illustrative routes of administration include transdermal, percutaneous, intravenous, intramuscular, intranasal, buccal, intrathecal, intracerebral, or intrarectal routes. The route of administration may be varied in any way, limited by the physical properties of the compounds being employed and the convenience of the subject and the caregiver.

As one skilled in the art will appreciate, suitable formulations include those that are suitable for more than one route of administration. For example, the formulation can be one that is suitable for both intrathecal and intracerebral administration. Alternatively, suitable formulations include those that are suitable for only one route of administration as well as those that are suitable for one or more routes of administration, but not suitable for one or more other routes of administration. For example, the formulation can be one that is suitable for oral, transdermal, percutaneous, intravenous, intramuscular, intranasal, buccal, and/or intrathecal administration but not suitable for intracerebral administration.

The inert ingredients and manner of formulation of the pharmaceutical compositions are conventional. The usual methods of formulation used in pharmaceutical science may be used here. All of the usual types of compositions may be used, including tablets, chewable tablets, capsules, solutions, parenteral solutions, intranasal sprays or powders, troches, suppositories, transdermal patches, and suspensions. In general, compositions contain from about 0.5% to about 50% of the compound in total, depending on the desired doses and the type of composition to be used. The amount of the compound, however, is best defined as the “effective amount”, that is, the amount of the compound which provides the desired dose to the patient in need of such treatment. The activity of the compounds employed in the compositions and methods disclosed herein are not believed to depend greatly on the nature of the composition, and, therefore, the compositions can be chosen and formulated primarily or solely for convenience and economy.

Capsules are prepared by mixing the compound with a suitable diluent and filling the proper amount of the mixture in capsules. The usual diluents include inert powdered substances (such as starches), powdered cellulose (especially crystalline and microcrystalline cellulose), sugars (such as fructose, mannitol and sucrose), grain flours, and similar edible powders.

Tablets are prepared by direct compression, by wet granulation, or by dry granulation. Their formulations usually incorporate diluents, binders, lubricants, and disintegrators (in addition to the compounds). Typical diluents include, for example, various types of starch, lactose, mannitol, kaolin, calcium phosphate or sulfate, inorganic salts (such as sodium chloride), and powdered sugar. Powdered cellulose derivatives can also be used. Typical tablet binders include substances such as starch, gelatin, and sugars (e.g., lactose, fructose, glucose, and the like). Natural and synthetic gums can also be used, including acacia, alginates, methylcellulose, polyvinylpyrrolidine, and the like. Polyethylene glycol, ethylcellulose, and waxes can also serve as binders.

Tablets can be coated with sugar, e.g., as a flavor enhancer and sealant. The compounds also may be formulated as chewable tablets, by using large amounts of pleasant-tasting substances, such as mannitol, in the formulation. Instantly dissolving tablet-like formulations can also be employed, for example, to assure that the patient consumes the dosage form and to avoid the difficulty that some patients experience in swallowing solid objects.

A lubricant can be used in the tablet formulation to prevent the tablet and punches from sticking in the die. The lubricant can be chosen from such slippery solids as talc, magnesium and calcium stearate, stearic acid, and hydrogenated vegetable oils.

Tablets can also contain disintegrators. Disintegrators are substances that swell when wetted to break up the tablet and release the compound. They include starches, clays, celluloses, algins, and gums. As further illustration, corn and potato starches, methylcellulose, agar, bentonite, wood cellulose, powdered natural sponge, cation-exchange resins, alginic acid, guar gum, citrus pulp, sodium lauryl sulfate, and carboxymethylcellulose can be used.

Compositions can be formulated as enteric formulations, for example, to protect the active ingredient from the strongly acid contents of the stomach. Such formulations can be created by coating a solid dosage form with a film of a polymer which is insoluble in acid environments and soluble in basic environments. Illustrative films include cellulose acetate phthalate, polyvinyl acetate phthalate, hydroxypropyl methylcellulose phthalate, and hydroxypropyl methylcellulose acetate succinate.

Transdermal patches can also be used to deliver the compounds. Transdermal patches can include a resinous composition in which the compound will dissolve or partially dissolve; and a film which protects the composition, and which holds the resinous composition in contact with the skin. Other, more complicated patch compositions can also be used, such as those having a membrane pierced with a plurality of pores through which the drugs are pumped by osmotic action.

As one skilled in the art will also appreciate, the formulation can be prepared with materials (e.g., actives excipients, carriers (such as cyclodextrins), diluents, etc.) having properties (e.g., purity) that render the formulation suitable for administration to humans. Alternatively, the formulation can be prepared with materials having purity and/or other properties that render the formulation suitable for administration to non-human subjects, but not suitable for administration to humans.

### Methods of Generating Recombinant Rotavirus

In another aspect of the current disclosure, methods of generating rotavirus in vitro are provided. In some embodiments, the methods comprise introducing a composition comprising a polynucleotide comprising a sequence encoding a recombinant rotavirus protein, wherein the polynucleotide encodes a positive sense viral transcript; allowing the cell to express one or more rotavirus proteins selected from VP1, VP2, VP3, VP4, VP6, VP7, NSP1, NSP2, NSP3, NSP4, and NSP5; incubating the cells for a sufficient time to produce rotavirus; and harvesting virus produced by the cells to generate rotavirus in vitro.

### Cells

The inventors disclose herein cells comprising the disclosed compositions, which may also be used in the disclosed methods and systems. Accordingly, in another aspect of the current disclosure, cells are provided. In some embodiments, the cells comprise a composition comprising a polynucleotide comprising a sequence encoding a recombinant rotavirus protein, wherein the polynucleotide encodes a positive sense viral transcript. In some embodiments, the cells are selected from MA-104 cells, Vero cells, and BHK-1 cells.

Rotavirus vaccine strains have been traditionally grown using Vero cells. This method of producing rotavirus has been found to be suitable for generation of rotavirus for administration to subjects. Therefore, in some embodiments, the cells are Vero cells.

In some embodiments, the cells disclosed herein further comprise a heterologous RNA polymerase, wherein the heterologous RNA polymerase binds to the promoter in the disclosed compositions and catalyzes sequence-dependent RNA polymerization based on the composition when the composition is introduced into the cell. As used herein, “heterologous RNA polymerase” refers to an RNA polymerase not present in the cell without introduction through molecular biological techniques, e.g., transduction, transfection, lipofection, etc. In some embodiments, the heterologous RNA polymerase comprises T7 bacteriophage RNA polymerase or T3 bacteriophage RNA polymerase, more commonly known as simply T7 polymerase and T3 polymerase, respectively.

Accordingly, in some embodiments, the cells further comprise T7 RNA polymerase or T3 RNA polymerase. In some embodiments, such cells are referred to as, e.g., BHK-T7 cells, because they are derived from BHK-1 cells, but express the heterologous RNA polymerase T7 bacteriophage RNA polymerase. Thus, as used herein, “BHK-T7 cells” are BHK-1 cells that express the heterologous RNA polymerase T7 bacteriophage RNA polymerase.

### Methods of Eliciting an Immune Response

The instant disclosure provides compositions, methods of making the same, and infectious particles. Therefore, in another aspect of the current disclosure, methods of eliciting an immune response are provided. In some embodiments, the methods comprise administering a pharmaceutical composition comprising an infectious particle comprising a composition comprising a polynucleotide comprising a sequence encoding a recombinant rotavirus protein, wherein the polynucleotide encodes a positive sense viral transcript.

In some embodiments, the methods comprise administering a pharmaceutical composition comprising an infectious particle made by transfecting cells with a composition comprising a polynucleotide comprising a sequence encoding a recombinant rotavirus protein, wherein the polynucleotide encodes a positive sense viral transcript.

In some embodiments, methods of eliciting an immune response are provided. In some embodiments, the methods of eliciting an immune response comprise administering a pharmaceutical composition comprising an infectious particle comprising a composition comprising a polynucleotide comprising a sequence encoding a recombinant rotavirus protein, wherein the polynucleotide encodes a positive sense viral transcript. In some embodiments, methods of eliciting an immune response comprise administering a pharmaceutical composition comprising an infectious particle made by transfecting cells with a composition comprising a polynucleotide comprising a sequence encoding a recombinant rotavirus protein, wherein the polynucleotide encodes a positive sense viral transcript.

### Methods of Vaccinating a Subject

In another aspect of the current disclosure, methods of vaccinating a subject against one or more pathogens are provided. In some embodiments, the methods comprise administering a pharmaceutical composition comprising an infectious particle comprising a composition comprising a polynucleotide comprising a sequence encoding a recombinant rotavirus protein, wherein the polynucleotide encodes a positive sense viral transcript. In some embodiments, the methods comprise administering a pharmaceutical composition comprising an infectious particle made by transfecting cells with a composition comprising a polynucleotide comprising a sequence encoding a recombinant rotavirus protein, wherein the polynucleotide encodes a positive sense viral transcript.

### Systems for Generating Recombinant Rotavirus

In another aspect of the current disclosure, systems for generating recombinant rotavirus are provided. In some embodiments, the systems comprise: (a) a composition comprising a polynucleotide comprising a sequence encoding a recombinant rotavirus protein, wherein the polynucleotide encodes a positive sense viral transcript; and (b) cells capable of expressing the compositions of (a).

### EXAMPLES

The following Examples are illustrative and should not be interpreted to limit the scope of the claimed subject matter.

### Example 1—Generation of a Novel Recombinant Rotavirus Based on RIX4414 Using Reverse Genetics

The inventors report the development of a reverse genetics system for the human rotavirus GTP[8] strain RIX4414. This invention allows us to modify the rotavirus strain formulating the widely used Rotarix vaccine and provides a path for generating modified RIX4414 strains that express capsid proteins of other infectious viruses, including norovirus. Used in leu of RIX4414, rotavirus vaccines formulated with RIX4414-expressing norovirus capsid proteins may provide immunological protection against the two most common causes of severe viral gastroenteritis in children: rotavirus and norovirus.

The RIX4414 reverse genetics system was developed as follows. Sequences for the eleven genome segments of the RIX44144 strain were predicted based on data available in NCBI GenBank (SEQ ID NOs: 1-11). The sequences were used to construct eleven pUC19-based pT7 transcription vectors, each expressing one of the RIX4414 plus-sense RNAs. The RIX4414 pT7 plasmids were used to produce recombinant RIX4414, generally following reverse genetics protocols previously described in A. A. Phillips et al. 2020. As before, the RIX4414 pT7 transcription plasmids and the pCMV-NP868R capping enzyme plasmid were transfected into BHK-T7 cells. Two days later, transfected BHK-T7 cells were overseeded with MA104 cells. Three days later, the BHK-T7/MA104 cell culture was overseeded with Vero cells (This is unique to RIX4414 reverse genetics system). Eight days later, the BHK-T7/MA104 cell mixture was freeze-thawed 3-times and used to amplify the recombinant viruses. When the cell culture reached complete infection, a lysate was prepared from the cells and rRIX4414 recovered by plaque isolation.

### Example 2—T7 Expression Plasmids for Producing a Recombinant Human G1P[8] Rotavirus Comprised of RIX44143 Sequences of the RV1 (Rotarix®, GSK) Vaccine Strain

RV1 (Rotarix®, GSK), the most widely used rotavirus vaccine, is formulated from the human G1P[8] virus RIX4414 (1,2). To protect against rotavirus gastroenteritis, >24 million children received the RV1 vaccine in 2021 (2). A challenge to rotavirus immunization efforts is that RV1, as well as other rotavirus vaccines, reach efficacies in low-income countries (50-64%) that can be significantly lower than in high- and moderate-income countries (85-98%) (3,4). The inventors report here the development of T7 expression plasmids that allow recovery of a recombinant RIX4414-like virus by reverse genetics. By this method, it may be possible to generate modified forms of the RV1 vaccine with improved performance in low-income countries.

The rotavirus genome consists of eleven segments of double-stranded RNA (5). The rotavirus strain RIX4414 (originally named 89-12) was isolated from a child with acute gastroenteritis in 1989 and serially passaged in cell culture to promote the introduction of attenuating mutations (6). In this study, the inventors designed eleven pT7 expression plasmids, each containing a cDNA sequence corresponding to one of the RIX4414 genome segments, using sequencing information for RIX4414 available in GenBank (Table 2). In cases where RIX4414 sequence information was missing, vis-a-vis, portions of the 5′ and 3′-untranslated regions, sequence information for the prototypic human GTP[8] Wa virus was used instead (Table 2). Because the original pT7/RIX4414 VP2 plasmid was not functional in the reverse genetics system, a modified plasmid was made with slight modifications in the RIX4414 VP2 coding region to include residues common to other human GT/4P[8] virus strains (e.g., Wa, KU (7), Odelia (8)) (Table 2). The RIX4414 cDNA sequences were positioned downstream of a T7 promoter and upstream of a hepatitis D virus ribozyme sequence in the pT7 plasmids (9, 10). The pT7 plasmids were generated by Azenta Life Sciences using a pUC19 backbone.

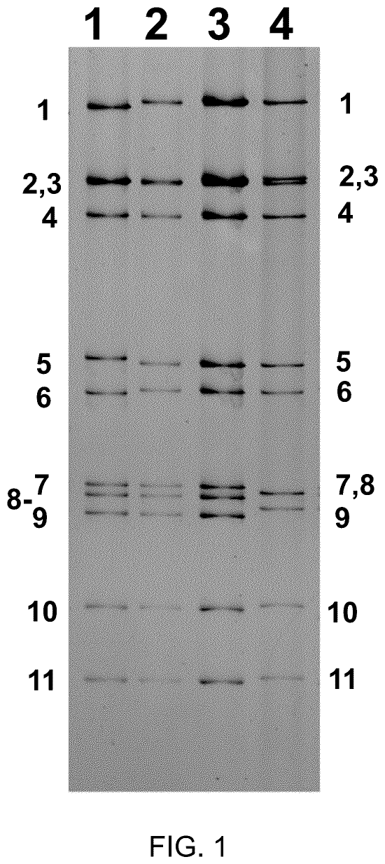

The RIX4414 pT7 plasmids supported the recovery of recombinant (r)RIX4414-like virus using a slightly modified reverse genetics procedure (11,12). Briefly, individual wells of a 12-well plate containing BHK-T7 cells were transfected with plasmid mixtures containing 0.8 μg of each RIX4414 T7 plasmid (except the NSP2 and NSP5 plasmids, which were 4.8 μg each), 1.6 μg of pCMV-NP868R RNA capping plasmid, and 1.6 g of pcDNA-T7 RNA polymerase plasmid. Two days later, the transfected BHK-T7 cells were overseeded with 105 MA104 cells/well. Three days later, the BHK-T7/MA104 cell culture was overseeded with 105 Vero cells/well. Eight days later, rRIX4414-like viruses contained in the cell lysates were amplified using Vero cells and plaque-isolated using MA104 cells (13). The RNA genome profile of the rRIX4414-like virus is presented in FIG. 1. The genome sequence of the rRIX4414-like isolate was confirmed by Nanopore sequencing (14). Based on sequence analysis, the rRIX4414-like virus has nucleotide and amino acid sequence identities of >99% with the RIX4414 vaccine virus, making the rRIX4414 reverse genetics system an ideal tool for investigating genetic changes that may improve RVT performance in low-income countries.

### REFERENCES

1. Ward R L, Bernstein D I. Rotarix: a rotavirus vaccine for the world. Clin Infect Dis. 2009 Jan. 15; 48(2):222-8.
2. UNICEF, 2022. Rotavirus Vaccine: Supply and Demand Update. UNICEF Supply Division, January 2022.
3. Jonesteller C L, Burnett E, Yen C, Tate J E, Parashar U D. Effectiveness of Rotavirus Vaccination: A Systematic Review of the First Decade of Global Postlicensure Data, 2006-2016. Clin Infect Dis. 2017; 65(5):840-850.
4. Bergman H, Henschke N, Hungerford D, Pitan F, Ndwandwe D, Cunliffe N, Soares-Weiser K. Vaccines for preventing rotavirus diarrhoea: vaccines in use. Cochrane Database Syst Rev. 2021 Nov. 17; 11(11):CD008521.
5. Trask S D, McDonald S M, Patton J T. Structural insights into the coupling of virion assembly and rotavirus replication. Nat Rev Microbiol. 2012 Jan. 23; 10(3):165-77. doi: 10.1038/nrmicro2673. Erratum in: Nat Rev Microbiol. 2014 January; 12(1):70.
6. Bernstein D I, Smith V E, Sherwood J R, Schiff G M, Sander D S, DeFeudis D, Spriggs D R, Ward R L. Safety and immunogenicity of live, attenuated human rotavirus vaccine 89-12. Vaccine. 1998 February; 16(4):381-7.
7. Komoto S, Fukuda S, Kugita M, Hatazawa R, Koyama C, Katayama K, Murata T, Taniguchi K. Generation of Infectious Recombinant Human Rotaviruses from Just 11 Cloned cDNAs Encoding the Rotavirus Genome. J Virol. 2019 Apr. 3; 93(8):e02207-18.
8. Kawagishi T, Nurdin J A, Onishi M, Nouda R, Kanai Y, Tajima T, Ushijima H, Kobayashi T. Reverse Genetics System for a Human Group A Rotavirus. J Virol. 2020 Jan. 6; 94(2):e00963-19.
9. Kanai Y, Komoto S, Kawagishi T, Nouda R, Nagasawa N, Onishi M, Matsuura Y, Taniguchi K, Kobayashi T. Entirely plasmid-based reverse genetics system for rotaviruses. Proc Natl Acad Sci USA. 2017 Feb. 28; 114(9):2349-2354.
10. Philip A A, Perry J L, Eaton H E, Shmulevitz M, Hyser J M, Patton J T. Generation of Recombinant Rotavirus Expressing NSP3-UnaG Fusion Protein by a Simplified Reverse Genetics System. J Virol. 2019 Nov. 26; 93(24):e01616-19.
11. Philip A A, Dai J, Katen S P, Patton J T. Simplified Reverse Genetics Method to Recover Recombinant Rotaviruses Expressing Reporter Proteins. J Vis Exp. 2020 Apr. 17; (158).
12. Philip A A, Patton J T. Generation of Recombinant Rotaviruses Expressing Human Norovirus Capsid Proteins. J Virol. 2022 Nov. 23; 96(22):e0126222.
13. Arnold M, Patton J T, McDonald S M. Culturing, storage, and quantification of rotaviruses. Curr Protoc Microbiol. 2009 November; Chapter 15:Unit 15C.3.
14. Faizuloev E, Mintaev R, Petrusha O, Marova A, Smimova D, Ammour Y, Meskina E, Sergeev O, Zhavoronok S, Karaulov A, Svitich O, Zverev V. New approach of genetic characterization of group A rotaviruses by the nanopore sequencing method. J Virol Methods. 2021 June; 292:114114.

Each of the above references are incorporated herein by reference.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

TABLE 2
RIX4414 sequences used in the generation of T7 expression plasmids

| | | Sources of RIX4414 sequences | | | | | |
|---|---|---|---|---|---|---|---|
| | | assembled into T7 expression plasmids | | | | | |
| | | | | 3′ UTR | | | |
| | | | | completed | | | |
| | | | | with RVA/ | | | |
| | RIX4414 sequence | | | Human-tc/ | | | |
| | reported in GenBank | | 5′ UTR | USA/Wa/ | Accession numbers | | Percent sequence |
| | (RVA/Human- | | completed with | 1974/G1P[8] | RIX4414 | | identity (nt/aa), T7 |
| | lab/USA/Rotarix_{—} | | RVA/Human- | or other | sequences in | Vaccine | plasmid RIX4414 |
| Genome | SSCRTV_00092/ | Full/partial | tc/USA/Wa/ | genotype | T7 expression | RIX4414 | versus RIX4414 |
| segment | 2016/G1P[X]) | sequence | 1974/G1P[8] | 1 strain | plasmids | (lab-strain) | lab-strain |
| VP1 | MF469220 | MF469220 | | | OR187571 | OR187592 | 100.00/100.00 |
| | | (full) | | | | | |
| VP2 | MF469221 | MF469221 | | | OR187573 | OR187593 | 99.78/99.32 |
| | | full (mutations | | | | | |
| | | S12N, N30D, | | | | | |
| | | P108S, K120R, | | | | | |
| | | I390T, V616T) | | | | | |
| VP3 | MF469222 | MF469222 | JX406749 | | OR187574 | OR187594 | 99.96/100.00 |
| | | (partial) | (Wa) | | | | |
| VP4 | JN849113 | JN849113 | | | OR187575 | OR187595 | 99.92/99.75 |
| | | (full) | | | | | |
| VP6 | MF469223 | MF469223 | | | OR187576 | OR187596 | 99.93/100.00 |
| | | (full) | | | | | |
| VP7 | MF469224 | MF469224 | | | OR187577 | OR187597 | 99.91/100.00 |
| | | (full) | | | | | |
| NSP1 | MF469215 | MF469215 | JX406751 | JX406751 | OR187578 | OR187598 | 99.87/100.00 |
| | | (partial) | (Wa) | (Wa) | | | |
| NSP2 | MF469216 | MF469216 | JX406754 | LC438386/ | OR187579 | OR187599 | 99.91/100.00 |
| | | (partial) | (Wa) | LC485138 | | | |
| | | | | (KU/Odelia) | | | |
| NSP3 | MF469217 | MF469217 | | JX406753 | OR187580 | OR187600 | 99.91/100.00 |
| | | (partial) | | (Wa) | | | |
| NSP4 | MF469218 | MF469218 | JX406756 | JX406756 | OR187581 | OR187601 | 100.00/100.00 |
| | | (partial) | (Wa) | (Wa) | | | |
| NSP5 | MF469219 | MF469219 | | | OR187582 | OR187602 | 99.85/100.00 |
| | | (full) | | | | | |

a Full, T7 expression plasmid contains a complete cDNA sequence for a RIX4414 genome segment matching the GenBank number. Partial, Complete rotavirus cDNA sequence in T7 expression plasmid was generated by combining the partial RIX4414 segment sequence reported by the GenBank number with the 5′ and/or 3′ UTR sequences of other Wa-like rotaviruses.
b VP2 protein encoded by T7 expression plasmid was engineered to contain residues common to other genotype C1 VP2 segments previously used in developing reverse genetics systems (7, 8).

We claim:

1. A composition comprising a polynucleotide comprising a sequence encoding a recombinant rotavirus protein, wherein the polynucleotide encodes a positive sense viral transcript, wherein the recombinant rotavirus protein is an RIX4414 strain rotavirus protein.

2.-4. (canceled)

5. The composition of claim 1, wherein the polynucleotide comprises any one of SEQ ID NOs 1-11.

6.-7. (canceled)

8. The composition of claim 5, wherein the polynucleotide further comprises a heterologous polynucleotide.

9.-16. (canceled)

17. The composition of claim 1, wherein the polynucleotide comprises a sequence encoding a cleavage site.

18.-21. (canceled)

22. The composition of claim 1, wherein the polynucleotide comprises a sequence encoding a linker.

23. (canceled)

24. A collection of polynucleotides, wherein each of the polynucleotides in the collection comprises a sequence encoding at least one RIX4414 strain rotavirus protein selected from VP1, VP2, VP3, VP4, VP6, VP7, NSP1, NSP2, NSP3, NSP4, and NSP5, wherein the polynucleotides of the collection encode each of the VP1, VP2, VP3, VP4, VP6, VP7, NSP1, NSP2, NSP3, NSP4, and NSP5 proteins, wherein each of the sequences encoding one rotavirus protein are operably linked to a promoter.

25. The collection of claim 24, wherein the sequences encoding at least one rotavirus protein selected from VP1, VP2, VP3, VP4, VP6, VP7, NSP1, NSP2, NSP3, NSP4, and NSP5 comprise SEQ ID NOs: 1-11, respectively.

26. (canceled)

27. An infectious particle comprising the composition of claim 1.

28.-30. (canceled)

31. A pharmaceutical composition comprising the infectious particle of claim 27.

32. (canceled)

33. A method of eliciting an immune response to one or more microorganism in a subject, the method comprising:
administering the pharmaceutical composition of claim 31 to a subject to elicit an immune response to the one or more microorganism.

34.-40. (canceled)

41. A cell comprising the composition of claim 1.

42. A cell comprising the collection of claim 24.

43. The cell of claim 41, wherein the cell is an MA-104 cell, a Vero cell or a BHK-1 cell.

44. The cell of claim 42, wherein the cell is an MA-104 cell, a Vero cell or a BHK-1 cell.

45. (canceled)

46. A method of generating a rotavirus in vitro comprising: introducing the composition of claim 1 into a cell; allowing the cell to express one or more rotavirus proteins selected from VP1, VP2, VP3, VP4, VP6, VP7, NSP1, NSP2, NSP3, NSP4, and NSP5; incubating the cells for a sufficient time to produce rotavirus; and harvesting virus produced by the cells to generate the rotavirus in vitro.

47. A method of generating a rotavirus in vitro comprising: introducing the collection of claim 24 into a cell; incubating the cells for a sufficient time to produce rotavirus; and harvesting virus produced by the cells to generate the rotavirus in vitro.

48. The method of claim 46, wherein the cell is selected from an MA-104 cell, a Vero cell, and a BHK-1 cell.

49. The method of claim 47, wherein the cell is selected from an MA-104 cell, a Vero cell, and a BHK-1 cell.

50.-52. (canceled)

53. A system for generating recombinant rotavirus comprising:
(a) the composition of claim 1; and
(b) cells capable of expressing the compositions of (a).

54. A system for generating recombinant rotavirus comprising:
(a) the collection of claim 1; and
(b) cells capable of expressing the collection of (a).

55.-58. (canceled)


SEQUENCE LISTING (26 sequences)

SEQ ID NO: 1 - DNA, 3302 bp, synthetic construct
  source 1..3302: mol_type = other DNA
ggctattaaagctgtacaatggggaagtacaatctaatcttgtcagaatatttatcatttgtttacaattcacaatctgcagttcaaataccaatttactattcttctaattcagaattagaaaagagatgtatcgaatttcatgctaaatgtgttgacagttctaagaaaggtctgtcattaaaacctttattcgaagaatataaagatgtaataaataatgcaactttactatctatattatcatattcttatgacaaatacaacgctgtagaacgaaagctagtcaattatgctaaaggtaaaccattagaggctaatttaacggcgaacgagattgattatgaaaataataaaataacttctgaattgtttcagtcggccgaagaatatactgattcattaatggatcctgctatattaacttcgttatcttcaaatttaaatgcagtcatgttttggttagaacgacattcgaatgacgttgctgatgcaaacaaaatttataaacgtagactggatctatttatcatagtagcatctacaataaataaatatggagtgcctagacataatgaaaaatacagatatgaatacgaggtgatgaaggataaaccgtattacttagtaacatgggctaactcatctatagaaatgcttatgtcagtgttttcacatgaagattatttaatagcaaaagagttaataatattgtcatattcaaatagatcgacgttagctaaactagtttcatctccaatgtcgatattagttgcattaatagatatcaatggtacgtttattacaaatgaagagttagagcttgaattttcagataaatatgtcaaagcaattgtacctgatcaaattttcgatgaattacaggaaatgatcgacaatatgagaaaagctggtttagtagatataccaagaatgattcaagaatggttagttgattgttcattagagaaatttacactgatgtcaaaaatttattcttggtcatttcatgttggatttagaaaacaaaagatgattgacgcagcattagaccaattgaagacagagtacactgaagatgtagatggtgagatgtacaatgagtatacaatgttaattagagatgaaatagtcaagatgctagaagtaccagttaaacatgatgatcatctacttcgcgattcagaattagccggattgttatcaatgtcatcagcttcaaacggtgaatcaaggcagcttaaatttggtcgtaagacaatattttcaactaagaaaaatatgcatgtcatggatgatattgcacatggaagatatactccgggtgtcattcctccagtaaacgtagatagaccaattccattaggtcgtagagatgttcctggacgaagaacaagaattatattcatattaccatatgaatactttattgcgcagcacgctgtcgtagaaaaaatgttatcatacgcaaagcatactagagagtacgcagaattttactcgcagtcaaatcaactattatcatatggtgatgttacaagattcttatccagtaattctatggtattatacacagacgtttcacaatgggattcgtcacaacataacacacagccatttagaaaaggaataattatgggtttagatatgttatccaatatgactaatgatccaaaagtagtacaaacgttaaatttatataaacaaacacaaattaatctcatggattcatacgttcaaatacctgatggtaatgtaattaaaaagattcagtatggtgctgttgcttcaggagaaaaacaaactaaggcagctaattctatagctaacttagcactcattaaaacggtattgtcaaggattgcaaataagtattcttttataaccaaaataatcagagtcgatggtgatgataattatgcagtgctacaatttaacaccgacgtcactaaacagatggtccaagatgtgtcaaacgatgtgagatatatatattctagaatgaatgctaaagttaaagcattggtatctacagtcggtattgaaatagcaaaaagatatatagcaggaggaaaaatatttttcagagctggtataaacttattaaataatgagaaacgtgggcaaagtacacaatgggatcaagcagctattttatattcaaactacattgttaacaaattacgaggatttgagactgatagagaatttatattaactaaaattatacaaatgacatctgtagccattactggatcactaaggttatttccgtcagaacgagtgttaactactaattctacattcaaagtatttgactcagaagatttcattatagaatatgggacaactaacgatgaagtatatatacaaagagcatttatgtcactgtctagtcaaaagtcaggaatagctgatgaaatagcttcttcacaaacatttaaaaattatgttaacaaattatctgaccaattattaatatcaaaaaacgtaattgtatccaaaggtatagctgtaacagaaaaggcgaaattgaattcatatgcaccagtttatctagaaaaacgtcgtgcgcaaatatcagcgttattaactatgttgcagaaaccagtgtcatttaaatcgaataaaattactattaatgacattttgcgtgacataaaaccattttttgtcacttctgaagctaatttgccaattcaatacagaaaatttatgccaacactacctaataatgtccaatatgttatacaatgcataggatcgaggacgtatcaaatagaagatagtgggtcaaaatcatcgatttcgaagttaatatcaaaatattcagtttacaaaccgtcgattgaagaactatataaagttatatctttaagagaacaagaaatacagttgtatttagtttcattaggagttccaccagttgatgcaggcacgtacgttgggtcaagaatatattcgcaggataagtataaaatattagagtcttacgtatataatttactatcaattaattatggatgttatcaactattcgattttaattctccagatttagagaaacttatacgaattccttttaaaggcaagataccagccgtaacatttatattgcatctttatgctaaacttgaaataataaactatgctattaagaatggagcttggattagtttgttttgtaactacccaaaatctgaaatgattaaattatggaagaaaatgtggaatataacagcattacggtctccctacactagtgcgaacttctttcaagattagagtgcttagatgtgacc

SEQ ID NO: 2 - DNA, 2717 bp, synthetic construct
  source 1..2717: mol_type = other DNA
ggctattaaaggctcaatggcgtacaggaagcgcggagctaaacgtgaaaacttaccacaacaaaatgaacgtctgcaagaaaaagaaattgaaaaagatgtggatgtaactatggaaaataaaaataataatagaaagcagcaattatctgataaagtactatcacaaaaagaggaaataataactgatgcacaagatgatattaaaatagctggtgagattaaaaaatcatcaaaagaagagtcaaaacagttgctcgaaatattaaaaacgaaagaagaccatcagaaagaaatacagtatgaaattctacaaaaaacgataccgacttttgaatcaaaagaatcaattttgaaaaaattagaagatataagaccggagcaagctaagaagcaaatgaaattgtttagaatatttgaaccaaaacaattaccaatctatcgagcaaatggtgagaaagaattgagaaatagatggtattggaaattgaaaaaggatacgctgccagacggagattatgacgtacgagaatattttttaaatttatatgatcagatcctgatagaaatgccagattatttactactgaaagatatggctgtagaaaataaaaattctagagatgctggtaaagttgtagattctgaaacagcaaatatttgtgatgctatatttcaagatgaagagacagagggagttgtcagaagattcattgcagatatgagacaacaggttcaggctgatagaaatattgtcaattatccatcaattttacatccaattgatcacgcatttaatgaatattttctaaatcatcaattagtcgaaccactaaataatgaaatcatttttaattatataccagaaagaataaggaatgatgttaactatattttgaatatggatatgaatttgccatcaacagcaagatatattagaccgaacttgttgcaagatagactaaatttacatgataattttgaatcattatgggacacaataactacatcaaattatatactagccagatcagttgtgcctgatttgaaggaaaaagaattagtttcaactgaagctcagatacagaaaatgtctcaagatttacaacttgaagctttaacgatacaatctgaaacgcagtttcttgctggcataaattcacaagcagcaaatgattgttttaaaacattgatagcagctatgttaagccagcgtacaatgtcattagattttgtaaccacgaattatatgtcacttatatctggtatgtggctattgaccgttataccaaatgacatgtttcttcgtgaatcattagtcgcatgcgaattggccataataaatactatagtttatccagcatttggaatgcaaagaatgcattatagaaatggtgatccccagactccgtttcaaatagcagaacagcaaatacaaaattttcaagtagctaattggttacattttattaataataatagatttaggcaagttgttattgatggagtgttaaatcaaacacttaacgataatattaggaacggacaagttattaatcagttaatggaagcattaatgcagctatctagacaacaatttccgactatgccagttgattataaaagatcaatacaaagaggaatattactattatctaacaggttaggtcagttagttgatttaacaagattagtatcatataattatgaaactctaatggcttgtgtaactatgaatatgcaacatgttcaaactctcacgaccgaaaaattacaattgacttctgtcacatctttatgtatgttaattggaaatactacagtaattccaagtccacaaacattatttcactattataacataaatgtaaattttcattcaaattataacgaacgaattaacgacgcagtggctatcattacggctgctaacagactaaatttatatcagaaaaaaatgaaatcaatagttgaggattttttgaaaagattgcaaatttttgatgtaccacgagtaccagatgaccaaatgtacaggttgagagatagacttaggttattaccagttgaaagacgaagacttgatatatttaatttaatattaatgaatatggagcagatcgaacgagcttcagataaaattgctcaaggagtaataattgcttatagagatatgcaactagaaagagatgagatgtatggatatgtcaacattgctagaaatctcgatggatatcaacaaattaacctagaggagttgatgagaactggagactatgggcaaattactaatatgttattaaacaatcagcctgtagctttagtaggggcattaccatttgtaacggattcttcagttatatcactcattgcaaaattagatgctacagtttttgctcaaatagttaaacttagaaaagtggacactttaaaaccaatattgtataagataaattctgattctaatgatttctacttagttgcaaattatgattggataccaacttcaaccacaaaagtctataaacaagtaccacaaccttttgatttcagagcgtcaatgcatatgttaacgtctaatttgacttttacagtttattctgatttattatctttcgtttctgcagacacggttgaacccattaacgcagttgcttttgacaatatgcgcattatgaacgaactgtaaacgccaaccccactgtggagatatgacc

SEQ ID NO: 3 - DNA, 2591 bp, synthetic construct
  source 1..2591: mol_type = other DNA
ggctattaaagcagtactagtagtgcgttttacctctgatggtgtaagcatgaaagtattagctttaagacacagtgtggctcaggtgtatgcagatactcaaacctatttacatgatgattcaaaagatgagtatgaaaatgcatttctcatttctaatctaactacacataatatattatacctgaattacagccttaaaacattaaaaatattaaataaatcaggtgtagcagcagttgaagtacaatctccagatgaattatttgcgttaataaggtgtaattttacgtacgattatgagaacaacatagtttatttacatgattattcatattatactaacaatgaaataagaacagatcaacattggataactaaaactgatataattgactatctactacctggatggaaattaacgtatgtaggctataatggaaagaatacacgaggtcactataatttttcatttatatgtcaaaatgcagctacagatgatgatataataattgaatatatatactctaatgagttagactttcaaaattttctgttgagaaaaattaaagagagaatgaccacatctcttccaattgctagattatcaaatcgtgtgtttagagataaattatttccatctattgtaaacatacataaaaaagtgataaatgttgggccgaggaatgaatctatgttcacattcctaaattttccaactattaagcaattttcaaacggtgcgtatattgtgaagcatactattaaattgaagcaggagaaatggttgggtaaaagagtatcacaatttgatatcggacaatataaaaacatgttaaatgtggtcactactatttactattactataatttatactattcgaaacctataatatacatgcttggttcagctccatcttattggatttatgatattaaacagtattctgattttacatttgaaacatgggatccattagatacttcgtattctacaacacatcataaagaattattttttgagaaggatgtcaataagcttaaagataattcaattttatatatagatataagaactgatagaggtaatatggattggaaagagtggagaaaaatagtagaacaacaaactgttagcaatctaaatattgcatataaatatctatcaacaggaaaagcaaaggtgtgttgtgttaaattaactgctatggacctagaactaccaataacagcaaaattacttcatcatccaactactgaagttaggtcagaattttatgcaatattggatgtatgggatattattaccattaaaagatttataccaaaaggtgtattttatgcttttataaacaatgtaaccacagagaatgtgtttatacaacctccttttaaattaaaaacgtcacctactgattatatagtggcattgtatgctttatccaatgacttcaattcaaggcaagatgttattaatttgattaataaacaaaaacaatcactcattactgttcgtataaataacacttttaaagacgaaccaaaagtaaattttaaaaatatttatgattggacgtttctgccaacagatttcgaacttaaggattcgataattacatcatatgatggttgtttaggcatattcggattatcaatatctttatcgtccaaacccaccggaaataatcatttatttataataaatggaactgataaatatgataaactagaccaatatgcaaatcatatgggtatttctagaagatcacatcaaattagattctcagagtctgcgacatcatattcgggatatatttttagagatctttcaaataataatttcaatctaataggtactaatgtggagaattcagtttcaggacatgcctataatgcattgatttactacagatataattatgcgtttgatcttaaaagatggatttatctacattcaattggaaaagtcgctgtagaaggtggaagatattatgaacatgcaccaattgaattgatatatgcatgccgatcagctaaagaatttgctatattacaggacgacttaacggcattgagatacgctaatgaaattgaagggtatataaacaaagtatatagtataacctatgctgacgatcccaattacttcataggaattaaatttaatagtataccttatgaatacgatgttaaaattccacatttgacactaggagtattatttatatctgataatatgatacatgatgtaatcacagtgctgaagaaaatgaagacagagctatttaaaatggaaattagcactagctatacttatatgttatctgataatatgtatgtagcaaatgctagtggtgtgttatcaacttattttaaattatataatatattctatagaaatcatatcacgtttggacaatcacgaatgtttatcccacacattacgcttagttttagcaacaaacagacggttagaatagaaagtacaaaactcagaattaattcaatttacttaagaaagattaaaggtgaaaccgtatttgatatgtctgagtgagctagaaacttaacacactagtcatgatgtgacc

SEQ ID NO: 4 - DNA, 2359 bp, synthetic construct
  source 1..2359: mol_type = other DNA
ggctataaaatggcttcgctcatttatagacaacttctcactaattcatattcagtagatttacatgatgaaatagagcaaattggatcagaaaaaactcagaatgtaactataaatccgggtccatttgcacagactagatatgctccagtcaattgggatcatggagagataaatgattcgactacagtagaaccaattttagatggtccttatcagccaactacatttactccacctaatgattattggatacttattaattcaaatacaaatggagtagtatatgaaagtacaaataatagtgacttttggactgcagtcgttgctattgaaccgcacgtcaacccagtagatagacaatatatgatatttggtgaaagcaagcaatttaatgtgagtaacgattcaaataaatggaagtttttagaaatgtttagaagcagtagtcaaaatgaattttataatagacgtacattaacttctgataccagacttgtaggaatatttaaatatggtggaagagtatggacatttcatggtgaaacaccgagagctactactgacagttcaagtactgcaaatttaaataatatatcaattacaattcattcagaattttacattattccaaggtcccaggaatctaaatgtaatgaatatattaataatggtctgccaccaattcaaaatactagaaatgtagttccattgccattatcatctagatcgatacagtataagagagcacaagttaatgaagacattatagtttcaaaaacttcattatggaaagaaatgcagtataatagggatattataattagatttaaatttggtaatagtattgtaaagatgggaggactaggttataaatggtctgaaatatcatataaggcagcaaattatcaatataattacttacgtgacggtgaacaagtaaccgcacacaccacttgttcagtaaatggagtgaacaattttagctataatggagggtttctacccactgattttggtatttcaaggtatgaagttattaaagagaattcttatgtatatgtagactattgggatgattcaaaagcatttagaaatatggtatatgttaratcattagcagctaatttaaattcagtgaaatgtacaggtggaagttattatttcagtataccagtaggtgcatggccagtaatgaatggtggcgctgtttcgttgcattttgccggagttacattatccacgcaatttactgattttgtatcattaaattcactacgatttagatttagtttgacagttgatgaaccacctttctcaatactgagaacacgtacagtgaatttgtatggattaccagccgctaatccaaataatggaaatgaatactacgaaatatcaggaaggttttcactcatttctttagttccaactaatgatgattatcagactccaattatgaattcagtgacggtaagacaagatttagagcgccaacttactgatttacgagaagaatttaactcattgtcacaagaaatagctatggcacaattgattgatttagcactgttgcctctagatatgttttccatgttttcaggaattaaaagtacaattgatttaactaaatcaatggcgactagtgtaatgaagaaatttagaaaatcaaaattagctacatcaatttcagaaatgactaattcattgtcagatgctgcttcatcagcatcaagaaacgtttctattagatcgaatttatctgcgatttcaaattggactaatgtttcaaatgatgtgtcaaacgtaactaattcattgaacgatatttcaacacaaacatctacaattagtaagaaacttagattaaaagaaatgattactcaaactgaaggaatgagctttgacgacatttcagcagctgtactaaaaacaaaaatagatatgtctactcaaattggaaaaaatactttacctgatatagttacagaagcatctgagaaatttattccaaaacgatcatatcgaatattaaaggatgatgaagtaatggaaattaatactgaaggaaaattctttgcatacaaaattaatacatttgatgaagtgccattcgatgtaaataaattcgctgaactagtaacagattctccagttatatcagcgataatcgattttaagacattgaaaaatttaaatgataattatggaatcactcgtacagaagcgttaaatttaattaaatcgaatccaaatatgttacgtaatttcattaatcaaaataatccaattataaggaatagaattgaacagttaatactacaatgtaaattgtgagaacgctattgaggatgtgacc

SEQ ID NO: 5 - DNA, 1356 bp, synthetic construct
  source 1..1356: mol_type = other DNA
ggcttttaaacgaagtcttcgacatggaggttctgtactcactgtcaaaaactcttaaagatgctagggacaaaattgttgaaggtacattatattctaacgttagcgatcttattcagcaattcaatcaaatgatagtaactatgaatggaaatgactttcagactggaggaattggtaatttacctgttagaaattggactttcgattttggtctattaggtacaacacttttgaacttggatgctaattatgttgagaatgcaagaactacaattgaatattttattgactttattgataatgtatgtatggatgaaatggcaagagaatctcaaagaaatggagtatcgccacaatctgaagcgttaagaaagctagcgggaattaaatttaagagaataaatttcgataattcatcagaatacatagaaaattggaacttacaaaatagaagacagcgcaccggatttgtttttcataaacctaacatttttccatactcagcttcatttactctaaatagatctcaaccaatgcatgataatttaatgggaaccatgtggcttaatgctggatcagaaattcaagtggctggatttgattactcatgcgccataaatgcaccagcgaacatacagcaatttgagcatatcgttcagcttaggcgcgcactgactacagctactataactttattacctgatgcagagagatttagttttccaagagtaattaattcagctgatggcgcgactacatggttctttaatccagttattctaagaccaaacaatgtagaggtagaatttttattgaatggacaaattattaatacatatcaggctagatttggtactatcatcgcaagaaattttgatacaattcgtttattatttcagttgatgcgtccacctaatatgacaccagctgttaatgcactatttccacaagcacaaccttttcagcaccatgcaacagttggacttacattacgtattgaatctgcggtttgtgaatcagtgcttgcggacgcgaatgaaactctgttagcaaatgtgaccgcggtgcgtcaagaatatgccataccagttggaccggtatttccaccaggcatgaattggactgaattgattactaactattcgccatctagagaagataacttgcaacgcgttttcacggtagcttccattagaagcatgttgattaagtgaggaccagactaaacatctggtatccaatcttagttagcatgtagctacatcaagtcattcagactcttcaagtaaggacatgatttcatgttcgctacgtagagtaactgtctgaatgatgtagtgagaggatgtgacc

SEQ ID NO: 6 - DNA, 1062 bp, synthetic construct
  source 1..1062: mol_type = other DNA
ggctttaaaagagagaatttccgtctggctaacggttagctccttttaatgtatggtattgaatataccacaattctaatctttctgatatcaattattctactcaactatatattaaaatcagtaactcgaataatggactacattatatatagatctttgttgatttatgtagcattatttgccttgacaagagctcagaattatgggcttaacttaccaataacaggatcaatggacactgtatacgctaactctactcaagaaggaatatttctaacatccacattatgtttgtattatccaactgaagcaagtactcaaattaatgatggtgaatggaaagactcattgtcacaaatgtttctcacaaaaggttggccaacaggatcagtctattttaaagagtattcaagtattgttgatttttctgtcgatccacaattatattgtgattataacttagtactaatgaaatatgatcaaaatcttgaattagatatgtcagagttagctgatttaatattgaatgaatggttatgtaatccaatggatataacattatattattatcaacaatcgggagaatcaaataagtggatatcaatgggatcatcatgtactgtgaaagtgtgtccactgaatacgcaaatgttaggaataggttgtcaaacaacaaatgtagactcgtttgaaatggttgctgagaatgagaaattagctatagtggatgtcgttgatgggataaatcataaaataaatttgacaactacgacatgtactattcgaaattgtaagaagttaggtccaagagagaatgtagctgtaatacaagttggtggctctaatgtattagacataacagcagatccaacgactaatccacaaactgagagaatgatgagagtgaattggaaaaaatggtggcaagtattttatactatagtagattatattaaccaaatcgtgcaggtaatgtccaaaagatcaagatcattaaattctgcagctttttattatagagtatagatatatcttagattagaattgttcgatgtgacc

SEQ ID NO: 7 - DNA, 1567 bp, synthetic construct
  source 1..1567: mol_type = other DNA
ggctttttttatgaaaagtcttgtggaagccatggctacttttaaagatgcttgttatcattacaaaagaattaataaattgaataatacagtcttaaaattaggagttaatgatacatggagaccgtcatctccaactaagtataaaggatggtgtttagattgctgtcaacatactgacttaacttattgtagcgggtgtaccatgtatcatgtttgtcaatggtgcagtcaatatggtagatgttttcttgatgatgaaccacatttattaaggatgagaacttttaaaaatgaagtgacaaaagacgatttaaagaatttaattgatatgtatgaaacactattccctatgaatcataaaatagtatgtagattcattaacaatacgagacaacataaatgtagaaacgaatgtataacacagtggtacaatcatttactaatgccaataacgttacaatccctgtcaattgaattagatggtgacgtttattacgtatttgggtattatgataacatgaataatattaatcaaactccattctcatttataaatttagtagatatatatgataaattattacttgatgatgtaaattttactagaatgtcattcttaccagcatctttacagcaagaatatgcacttagatatttttctaaatcaagatttataagcgaacaaaggaaatgtgttaatgattcacacttttcaattaatgtgttagagaacttacataacccaagtttcaaaatacagattacacgtaattgtagcgaactgtcttttggctggaatgaagcatgtaagcttgttaaaaatgtcagtgcgtattttaacatgctgaaaacatcacatattgaattttatagtgtttctaccagatgtagaatattcacacaatgtaaactcaaaatggcatctaaattaatcaaaccaaattatataacctcgaatcataaaacgttagctactgaggtacacaattgcaagtggtgttcagttaataacagttatactgtatggaatgattttaggattaagaaaatatatgataatatttttaactttctacgagctttagttaaatcaaatgctaatattggacattgttcatcacaggaaaagatatatgaatatgttgaagacgttctgaatgtgtgtgatgacgaaagatggaaagcgtcaataatggaaatttttaattgtctagaaccagtagagcttgatgacggcaagtatgttttatttaaccatgaaataaattgggatgtgattaatgtattggtccatagtattggtaaagtaccacaaatattaactttggagaatgttatcacaatcatgcaatctatcatatatgaatggtttgatataagatacatgaggaatacaccaatggttacatttacaattgataaattaagacgattatatacaggattaaagactgttgaatatgattctggtatatctgatattgaatagtaaaagaaagatcacgtttgccactacaagactccctgcactagagtagcgcctaggcagcatagaatgtgaacc

SEQ ID NO: 8 - DNA, 1059 bp, synthetic construct
  source 1..1059: mol_type = other DNA
ggcttttaaagcgtctcagtcgccgtttgagccttgcggtgtagccatggctgagctagcttgcttttgttatcctcatttagagaacgatagctataaatttattccttttaataatttagcaataaaatgtatgctgacagctaaagtagataaaaaagatcaagataaattttacaactctattgtgtatggaattgcgccaccaccacaatttaagaaacgttacaatactaatgataattcaagaggtatgaattatgaaacagttatgttcaataaggtggccattttaatctgtgaagcactcaattcaattaaagttacccaatctgatgttgcgaatgttctttcaagagtagtttctatcagacatttagaaaatttggtactaagaaaagaaaatcatcaagatgtattgtttcattcgaaagaactgctattaaaatctgttctaatagctattggtcaatcaaaggaaattgaaactactgctactgctgaaggaggagaaatagtctttcaaaatacagcattcactatgtggaaattaacgtatctagatcataaattaatgcctattttggatcagaacttccttgaatataagatcacactgaatgaagataagccgatctcagatatgtgtgttaaagaacttgttgctgaattaaggtggcagtataataaatttgctgtaataacacatggtaaaggtcactatagagttgttaaatattcatcagttgctaaccatgcggatagagtgttcgctacatataaaaataacgctaagagtggaaacgttactgacttcaatctattggaccaaagaataatttggcagaattggtatgcatttacgtcttcaatgaaacaaggtaatacacttgatgtatgtaaaaaactactcttccaaaaaatgaaacaagagaaaaatccatttaaaggactgtcaactgatagaaaaatggatgaagtttcacatgtcggaatttaattcgctttcgattcaagaatgatgatgacggagcaagaatagaaagcgctaatgtgacc

SEQ ID NO: 9 - DNA, 1074 bp, synthetic construct
  source 1..1074: mol_type = other DNA
ggcttttaatgcttttcagtggttgctgctcaagatggagtctactcagcagatggtaagctctattattaacacttcttttgaagctgcagttgttgctgccacttcaacattagaattaatgggtattcaatatgattacaatgaagtatttaccagagttaaaagtaaatttgattatgtgatggatgactctggtgttaaaaacaatcttttgggtaaagctataactattgatcaggcgttaaatggaaagtttggctcagctattagaaatagaaattggatgactgattctaaaacggttgctaaattagatgaagacgtgaataaacttagaatgacattatcttctaaaggaatcgaccaaaagatgagagtacttaatgcttgttttagtgtaaaaagaataccaggaaaatcatcatcaataattaaatgcactagacttatgaaggacaaaatagaacgtggagaagttgaggttcatgattcatatgttgatgagaaaatggaaattgatactattgattggaaatctcgttatgatcagttagaaaaaagatttgagtcactaaaacaaagagttaatgagaaatacaatacttggatacaaaaagcgaagaaagtaaatgaaaatatgtactctcttcagaatgtcatttcacaacagcaaaaccaaatagcagatcttcaacaatattgtaataaattggaagctgatttgcaaggcaaatttagttcattagtgtcatcagttgagtggtatctaagatctatggaattaccagatgatgtaaagaatgatattgaacagcagttaaattcaattgatttaattaatcccattaatgctatagatgatatcgaatcgttgattagaaatttaattcaagattatgacagaacgtttttaatgttaaaaggactgttgaagcaatgcaactatgaatatgtatatgagtagtcacataattaaaaatgttaaccatctacacatgaccctctatgagcacaatagttaaaagctaacactgtcaaaaacctaaatggcgataggggcgttatgtgacc

SEQ ID NO: 10 - DNA, 750 bp, synthetic construct
  source 1..750: mol_type = other DNA
ggcttttaaaagttctgttccgagagagcgcgtgcggaaagatggataagcttgccgacctcaactacacattgagtgtaatcactttaatgaatgacacattgcattctataattcaagatcctggaatggcgtatttttcatatattgcatctgttctaacagttttattcatattacataaagcttcaattccaaccatgaaaatagcattgaaaacatcaaaatgttcatataaagtgattaaatattgtatagtcacgatcattaatactcttttaaaattggctggatataaagagcaggttactacaaaagacgaaattgagcaacagatggacagaattgttaaagagatgagacgtcagctggatatgattgataaattaactactcgtgaaattgaacaggttgaattgcttaaacgtatacatgacaacctgataactagaccagttgacgtcatagatatgtcgaaggaattcaatcagaaaaacatcaaaacgctagatgaatgggagagtggaaaaaatccatatgaaccgtcagaagtgactgcatccatgtgagaggttgagttaccgtcgtctgtcttcggaagcggcggaactcttcaccgcaagccccattagacctgatgattgactgagaagccacagtcaatcatatcgcgtgtggctcagccttaatcccgtttaaccaatccagcgagtgttggacgttaatggaaggaatggtcttagtgtgacc

SEQ ID NO: 11 - DNA, 664 bp, synthetic construct
  source 1..664: mol_type = other DNA
ggcttttaaagcgctacagtgatgtctctcagcattgacgtgacgagtcttccctcaatttcttctagtatctttaaaaatgaatcgtcttctacaacgtcaactctttctggaaaatctattggtaggaacgaacagtatgtttcatcagatatcgaagcattcaataaatacatgttgtcgaagtctccagaggatattggaccatctgattctgcttcaaacgatccactcaccagtttttcgattagatcgaatgcagttaagacaaatgcagatgctggcgtgtctatggattcatcaacacaatcacgaccttcaagcaacgttgggtgcgatcaaatggatttctccctaactaaaggtattaatgttagtgctagtcttgattcatgtgtatcaatttcaactaaccataaaaaggagaaatctaaaaaggataaaagtaggaaacactacccaaaaattgaagcagattccgactctgaagattacgttttggatgattcagatagtgatgacggtaaatgtaagaattgtaaatataaaaagaaatattttgtattaagaatgaggatgaaacaagtcgcaatgcaattgatagaagatttgtaatgtcaacctgaggacacactagggagctccccactcccgttttgtgacc

SEQ ID NO: 12 - protein, 6 aa, synthetic construct
LVPRGS

SEQ ID NO: 13 - protein, 19 aa, synthetic construct
ATNFSLLKQAGDVEENPGP

SEQ ID NO: 14 - DNA, 18 bp, synthetic construct
  source 1..18: mol_type = other DNA
taatacgactcactatag

SEQ ID NO: 15 - DNA, 18 bp, synthetic construct
  source 1..18: mol_type = other DNA
aattaaccctcactaaag

SEQ ID NO: 16 - DNA, 1620 bp, Norwalk virus
  source 1..1620: mol_type = other DNA
atgaagatggcgtcgagtgacgccaacccatctgatgggtccgcagccaacctcgtcccagaggtcaacaatgaggttatggctctggagcccgttgttggtgccgctattgcggcacctgtagcgggccaacaaaatataattgacccctggattagaaataattttgtacaagcccctggtggagagtttacagtgtcccctagaaacgctccaggtgagatactatggagcgcgcccttgggccctgatttgaacccctatctttctcatttgtccagaatgtacaatggttatgcaggcggtttcgaagtgcaagtaatcctcgcggggaacgcgttcaccgccgggaaagttatatttgcagcagttccaccaaactttccaactgaaggcttaagccccagccaggttactatgttcccccatataattgtagatgttaggcaattggaacctgtgttgatccccctacctgatgttaggaataatttctatcattacaatcaatcacatgattctacccttaagttgatagcaatgttgtatacaccactcagggctaataatgccggggacgatgtcttcacagtctcttgtcgagttctcacgaggccatcccccgattttgatttcatattcttggtgccacccacagttgaatcaagaactaaaccattcaccgtcccaatcttaactgttgaggaaatgtccaattcaagattccccattcctttggaaaagttgtacacgggtcctagcagtgcttttgttgtccaaccacaaaatggcagatgcacgactgatggcgtgctcttaggtactacccagctgtcagctgtcaacatctgtaactttaggggggatgtcacccatattgtgggcagccatgattatacaatgaatctggcttcccaaaattggagcaattatgacccaacagaagaaatcccagcccccctgggaacaccagattttgtggggaagatccaaggcctgctcacccagaccacaagagcggatggctcgacccgtgcccacaaagctacagtgagcactgggagtgtccacttcactccaaagctgggtagtgttcaattcaccactgacacaaacaatgatttccaaactggccaaaacacgaaattcaccccagttggcgtcatccaggacggtgatcaccatcagaatgagccccaacaatgggtactcccaaattactcaggtagaactggtcataatgtgcacctggcccctgccgttgcccccacttttccgggtgagcaactccttttctttagatccactatgcccggatgtagcgggtatcccaacatgaatttggattgcctactcccccaggaatgggtgctgcacttctaccaggaagcagctccagcacaatccgatgtggctctgctgagatttgtgaatccagacacaggtagggttctgtttgagtgcaagctccataaatcaggctatatcacagtggctcacaccggcccgtatgacttggttatcccccccaatggttattttagatttgattcctgggtcaaccagttctacacacttgcccccatgggaaatggaacggggcgcaggcgtgcattataa

SEQ ID NO: 17 - DNA, 948 bp, Norwalk virus
  source 1..948: mol_type = other DNA
aaaccattcaccgtcccaatcttaactgttgaggaaatgtccaattcaagattccccattcctttggaaaagttgtacacgggtcctagcagtgcttttgttgtccaaccacaaaatggcagatgcacgactgatggcgtgctcttaggtactacccagctgtcagctgtcaacatctgtaactttaggggggatgtcacccatattgtgggcagccatgattatacaatgaatctggcttcccaaaattggagcaattatgacccaacagaagaaatcccagcccccctgggaacaccagattttgtggggaagatccaaggcctgctcacccagaccacaagagcggatggctcgacccgtgcccacaaagctacagtgagcactgggagtgtccacttcactccaaagctgggtagtgttcaattcaccactgacacaaacaatgatttccaaactggccaaaacacgaaattcaccccagttggcgtcatccaggacggtgatcaccatcagaatgagccccaacaatgggtactcccaaattactcaggtagaactggtcataatgtgcacctggcccctgccgttgcccccacttttccgggtgagcaactccttttctttagatccactatgcccggatgtagcgggtatcccaacatgaatttggattgcctactcccccaggaatgggtgctgcacttctaccaggaagcagctccagcacaatccgatgtggctctgctgagatttgtgaatccagacacaggtagggttctgtttgagtgcaagctccataaatcaggctatatcacagtggctcacaccggcccgtatgacttggttatcccccccaatggttattttagatttgattcctgggtcaaccagttctacacacttgcccccatgggaaatggaacggggcgcaggcgtgcattataa

SEQ ID NO: 18 - DNA, 432 bp, Norwalk virus
  source 1..432: mol_type = other DNA
actacccagctgtcagctgtcaacatctgtaactttaggggggatgtcacccatattgtgggcagccatgattatacaatgaatctggcttcccaaaattggagcaattatgacccaacagaagaaatcccagcccccctgggaacaccagattttgtggggaagatccaaggcctgctcacccagaccacaagagcggatggctcgacccgtgcccacaaagctacagtgagcactgggagtgtccacttcactccaaagctgggtagtgttcaattcaccactgacacaaacaatgatttccaaactggccaaaacacgaaattcaccccagttggcgtcatccaggacggtgatcaccatcagaatgagccccaacaatgggtactcccaaattactcaggtagaactggtcataatgtgcacctgtga

SEQ ID NO: 19 - DNA, 1623 bp, Norwalk virus
  source 1..1623: mol_type = other DNA
atgaagatggcgtctaaggacgccaacccatctgatgggtccacagccaacctcgtcccagaggtcaacaatgaggttatggctttggagcccgttgttggtgccgctattgcggcacctgtagcgggccaacaaaatgtaattgacccctggattagaaataattttgtacaagcccctggtggagagtttacagtatcccctagaaacgctccaggtgaaatactatggagcgcgcccttgggccccgatctgaatccctacctttctcatttggccagaatgtacaatggctatgcaggtggctttgaagtgcaggtaatccttgcggggaatgcgttcaccgccgggaaagtcatatttgcagcagtcccaccaaatttcccaactgaaggcttgagccccagccaggtcactatgttcccccatatagtagtagatgttaggcaactggaacctgtgttgatccccttacctgatgttaggaacaatttctatcactacaaccaatcaaatgaccccaccattaaactgatagcaatgctgtacacaccacttagggctaataatgctggggaagatgtcttcacagtctcttgtcgagtcctcacgaggccatcccccgattttgatttcatatttttggtgccacccacagttgagtcaagaactaaaccattcaccgtcccaattttaactgttgaggaaatgaccaattcaagattccccattcctttggaaaagttgttcacgggtcctagcggtgcctttgttgttcaaccacaaaatggcaggtgcacgactgatggcgtgctcttaggcactacccaactgtctcctgtcaacatctgcaccttcagaggggatgtcacccacattgcaggtactcataattacacaatgaatttggcttctcaaaattggaacaattatgacccaacagaagaaatcccagcccctctggggaccccagatttcgtgggaaggatccaaggcatgctcacccaaactacaaggggagatggctctacccgcggtcacaaagctacagtgagcactggggatgtccacttcactccaaagctgggcagtattcagttcaacactgatacaaacaatgattttgaaactggccaaaacacgaaattcaccccagtcggtgttgtccaggatggtaatggtacccaccaaaatgaacctcaacaatgggtgctcccaagctactcaggtagaactggtcataatgtacacctagcccccgccgtagcccccacttttccgggtgagcaacttcttttcttcaggtccactatgcccggatgcagcgggtaccctaacatgaatttggattgcctactcccccaagaatgggtgcagcacttctaccaagaggcagctccagcacaatctgatgtggctctgctaagatttgtgaatccagacacaggtagggttctatttgagtgcaagcttcataaatcaggctatgtcacagtggctcacactggccagcatgatttggtcatcccccccaatggctactttagatttgattcctgggtcaaccaattctacacacttgcccccctgggaaatgggacggggcgtagacgtgcattataa

SEQ ID NO: 20 - DNA, 951 bp, Norwalk virus
  source 1..951: mol_type = other DNA
aaaccattcaccgtcccaattttaactgttgaggaaatgaccaattcaagattccccattcctttggaaaagttgttcacgggtcctagcggtgcctttgttgttcaaccacaaaatggcaggtgcacgactgatggcgtgctcttaggcactacccaactgtctcctgtcaacatctgcaccttcagaggggatgtcacccacattgcaggtactcataattacacaatgaatttggcttctcaaaattggaacaattatgacccaacagaagaaatcccagcccctctggggaccccagatttcgtgggaaggatccaaggcatgctcacccaaactacaaggggagatggctctacccgcggtcacaaagctacagtgagcactggggatgtccacttcactccaaagctgggcagtattcagttcaacactgatacaaacaatgattttgaaactggccaaaacacgaaattcaccccagtcggtgttgtccaggatggtaatggtacccaccaaaatgaacctcaacaatgggtgctcccaagctactcaggtagaactggtcataatgtacacctagcccccgccgtagcccccacttttccgggtgagcaacttcttttcttcaggtccactatgcccggatgcagcgggtaccctaacatgaatttggattgcctactcccccaagaatgggtgcagcacttctaccaagaggcagctccagcacaatctgatgtggctctgctaagatttgtgaatccagacacaggtagggttctatttgagtgcaagcttcataaatcaggctatgtcacagtggctcacactggccagcatgatttggtcatcccccccaatggctactttagatttgattcctgggtcaaccaattctacacacttgcccccctgggaaatgggacggggcgtagacgtgcattataa

SEQ ID NO: 21 - DNA, 1623 bp, Norwalk virus
  source 1..1623: mol_type = other DNA
atgaagatggcgtcgagtgacgccaacccatctgatgggtccgcagccaacctcgtcccagaggtcaacaatgaggttatggctctggagcccgttgttggtgccgccattgcggcacctgtagcgggccaacaaaatgtaattgacccctggattagaaataattttgtacaagcccctggtggagagtttacagtgtcccctagaaacgctccaggtgaaatactatggagcgcgcccttgggccctgatctaaatccctacctatcccatttggccagaatgtacaatggttatgcaggtggttttgaagtgcaggtaattctcgcggggaacgcgttcaccgccgggaaggtcatatttgcagcagtcccaccaaattttccaactgaaggcttgagccccagccaggtcactatgttcccccatatagtagtagatgttaggcaattagaacctgtgttgattcccttacccgatgttaggaataatttctatcattacaatcaatcaaatgaccccaccattaagttgatagcaatgttgtatacaccacttagggctaataatgctggggatgatgtcttcacagtttcttgccgagttctcacgagaccatcccccgattttgatttcatatttctagtaccacccacagttgagtcaagaactaaaccattctctgtcccagttttaactgttgaggagatgaccaattcaagattccccattcctttggaaaagttgttcacgggtcccagcagtgcctttgttgtccaaccacaaaacggtaggtgcacgactgatggcgtgctcctaggcaccacccaactgtctcctgtcaacatctgcaccttcagaggagatgtcacccatatcacaggtagtcgtaactacacaatgaatttggcttctcaaaattggagcaattatgacccaacagaagaaatcccagcccctctaggaactccagattttgtggggaagattcaaggcgtgctcacccaaaccacaaggacagatggctcaacacgcggccacaaagccacagtgtacactgggagcgccgactttgctccaaaactgggtagagttcaatttggaactgacacagaccatgattttgaagctaaccaaaacacaaaattcaccccagttggtgtcatccaagatggtagcaccacccaccgaaatgaaccccaacagtgggtgctcccaagttactcaggcagaaatactcctaatgtgcatctggcccccgctgtagcccccacttttccgggtgagcaacttctcttcttcagatccaccatgcccggatgcagcgggtaccccaacatggatttggactgtctgctcccccaggaatgggtgcagtacttctaccaagaggcagccccagcacaatctgatgtggctctgctaagatttgtgaatccagacacaggtagggttttgtttgagtgtaagcttcataaatcaggctatgttacagtggctcacactggccaacatgatttggttatcccccccaatggttattttaggtttgattcctgggtcaaccagttttacacgcttgcccccatgggaaatggaacggggcgtagacgtgcactataa

SEQ ID NO: 22 - DNA, 951 bp, Norwalk virus
  source 1..951: mol_type = other DNA
aaaccattctctgtcccagttttaactgttgaggagatgaccaattcaagattccccattcctttggaaaagttgttcacgggtcccagcagtgcctttgttgtccaaccacaaaacggtaggtgcacgactgatggcgtgctcctaggcaccacccaactgtctcctgtcaacatctgcaccttcagaggagatgtcacccatatcacaggtagtcgtaactacacaatgaatttggcttctcaaaattggagcaattatgacccaacagaagaaatcccagcccctctaggaactccagattttgtggggaagattcaaggcgtgctcacccaaaccacaaggacagatggctcaacacgcggccacaaagccacagtgtacactgggagcgccgactttgctccaaaactgggtagagttcaatttggaactgacacagaccatgattttgaagctaaccaaaacacaaaattcaccccagttggtgtcatccaagatggtagcaccacccaccgaaatgaaccccaacagtgggtgctcccaagttactcaggcagaaatactcctaatgtgcatctggcccccgctgtagcccccacttttccgggtgagcaacttctcttcttcagatccaccatgcccggatgcagcgggtaccccaacatggatttggactgtctgctcccccaggaatgggtgcagtacttctaccaagaggcagccccagcacaatctgatgtggctctgctaagatttgtgaatccagacacaggtagggttttgtttgagtgtaagcttcataaatcaggctatgttacagtggctcacactggccaacatgatttggttatcccccccaatggttattttaggtttgattcctgggtcaaccagttttacacgcttgcccccatgggaaatggaacggggcgtagacgtgcactataa

SEQ ID NO: 23 - DNA, 2055 bp, Severe acute respiratory syndrome coronavirus 2
  source 1..2055: mol_type = other DNA
gtgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgt

SEQ ID NO: 24 - DNA, 567 bp, Severe acute respiratory syndrome coronavirus 2
  source 1..567: mol_type = other DNA
ttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaact

SEQ ID NO: 25 - DNA, 867 bp, Severe acute respiratory syndrome coronavirus 2
  source 1..867: mol_type = other DNA
ggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataaca

SEQ ID NO: 26 - DNA, 1980 bp, Severe acute respiratory syndrome coronavirus 2
  source 1..1980: mol_type = other DNA
gtgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacaca